(19) United States
(12) Patent Application Publication
(10) Pub. No.: US 2026/0347809 A1
(43) Pub. Date: Nov. 5, 2026

(54) ESTERASES AND RELATED METHODS

(71) Applicant: Flagship Pioneering Innovations VII, LLC, Cambridge, MA (US)

(72) Inventors: Pengfei TIAN, Boston, MA (US); Trine Holst SORENSEN, Boston, MA (US); Stephen Merritt JONES, Worcester, MA (US); Molly Krisann GIBSON, Medford, MA (US); Alexandra Rachael SNEIDER, Cambridge, MA (US); Brett Adam BOGHIGIAN, Wellesley, MA (US)

(21) Appl. No.: 19/690,227

(22) Filed: May 28, 2026

Related U.S. Application Data
(63) Continuation of application No. PCT/US2024/060038, filed on Dec. 13, 2024.
(60) Provisional application No. 63/610,668, filed on Dec. 15, 2023.

(51) Int. Cl. C12N 9/18; C08G 63/183; C08G 63/78; C08J 11/10

(52) U.S. Cl. CPC C12N 9/18; C08G 63/183; C08G 63/78; C08J 11/105; C08J 2367/02

(57) ABSTRACT

Provided herein are esterases (e.g., PETases) (and functional fragments, functional variants, and domains thereof), nucleic acid molecules encoding the same, and compositions comprising the same. The disclosure further relates to methods of utilizing the esterases (e.g., PETases) (or nucleic acid molecules encoding the same), including, e.g., in methods of degrading polyester containing products (e.g., plastic products).

### RELATED APPLICATIONS

This application claims priority to U.S. Ser. No. 63/610,668, filed Dec. 15, 2023, and is a continuation of International Application No. PCT/US2024/060038 filed Dec. 13, 2024, the entire contents of each of which is incorporated herein by reference.

### SEQUENCE LISTING

The instant application contains a Sequence Listing which has been submitted electronically in XML format and is hereby incorporated by reference in its entirety. Said XML copy, created on May 26, 2026, is named 62801.20US01_SL.xml and is 63,059 bytes in size.

### 1. FIELD

Provided herein are, inter alia, esterases (e.g., PETases) (and functional fragments, functional variants, and domains thereof), nucleic acid molecules encoding the same, and compositions comprising the same. The disclosure further relates to, inter alia, methods of utilizing the esterases (e.g., PETases) (or nucleic acid molecules encoding the same), including, e.g., in methods of degrading a polyester containing products (e.g., plastic products).

### 2. BACKGROUND

Polyethylene terephthalate (PET) is a linear semicrystalline thermoplastic polymer polyester. PET is commonly used in plastic products, including, e.g., textiles (e.g., clothing, bedding, carpets, upholstery, etc.) and packaging (e.g., plastic bottles (e.g., soda bottles), food jars, films, etc.). PET is produced from the polycondensation of ethylene glycol (EG) and terephthalic acid (TPA) through, e.g., an esterification reaction between TPA and EG or a trans-esterification reaction between EG and dimethyl terephthalate. PETases are a class of hydrolases (e.g., esterases) that catalyze the hydrolysis of PET into one or more product components, including, e.g., product mono-2-hydroxyethyl terephthalate (MHET).

### 3. SUMMARY

Provided herein are, inter alia, esterases (e.g., PETases) and polynucleotides encoding the same; fusions and conjugates comprising an esterase (e.g., PETase); methods of manufacturing; compositions; and methods of use including, e.g., methods of hydrolyzing a terephthalic acid ester, hydrolyzing a polyester, and degrading a polyester containing product (e.g., a plastic product).

Accordingly, in one aspect provided herein are recombinant esterases (e.g., a PETases) (or a functional fragments, functional variants, or domains thereof) that comprise an amino acid sequence at least 85%, 86%, 87%, 88%, 89%, 90%, 91%, 92%, 93%, 94%, 95%, 96%, 97%, 98%, 99%, or 100% identical to the amino acid sequence of any esterase (e.g., a PETase) set forth in Table 2 or set forth in any one of SEQ ID NOS: 2-12.

In some embodiments, the amino acid sequence of the esterase (e.g., PETase) is less than 100% (e.g., less than 99%, 98%, 97%, 96%, 95%, 94%, 93%, 92%, 91%, or 90%) identical to the amino acid sequence set forth in SEQ ID NO: 1.

In some embodiments, the amino acid sequence of the esterase (e.g., PETase) comprises at least one (e.g., 2, 3, 4, 5, 6, 7, 8, 9, 10, 11, 12, 13, 14, 15, 16, 17, 18, 19, 20, 21, 22, 23, 24, 25) amino acid variation (e.g., substitution) relative to the amino acid sequence set forth in SEQ ID NO: 1.

In some embodiments, the esterase has PETase activity. In some embodiments, esterase is a PETase. In some embodiments, the esterase exhibits enhanced esterase activity relative to a reference esterase (e.g., PETase) (e.g., a reference esterase (e.g., PETase) comprising the amino acid sequence set forth in SEQ ID NO: 1) (e.g., at specified conditions) (e.g., as determined as described in Example 2). In some embodiments, the esterase exhibits enhanced PETase activity relative to a reference PETase (e.g., a reference PETase comprising the amino acid sequence set forth in SEQ ID NO: 1) (e.g., at specified conditions) (e.g., as determined as described in Example 2).

In one aspect provided herein are recombinant esterases (e.g., a PETases) (or a functional fragments, functional variants, or domains thereof) that comprise an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, identical to the amino acid sequence set forth in SEQ ID NO: 1 and comprises one or more amino acid variation (e.g., substitution) relative to the amino acid sequence set forth in SEQ ID NO: 1.

In some embodiments, the recombinant esterase (e.g., a PETase) (or a functional fragment, functional variant, or domain thereof) comprises an amino acid variation (e.g., substitution) at one or more (e.g., 1, 2, 3, 4, 5, 6, 7, 8, 9, 10, 11, 12, 13, 14, 15, 16, 17, 18, 19, 21, 22, 23, 24, 25, 26, 27, 28, 29, 30, 31, 32, 33, 34, 35, 36) of amino acid positions S11, T14, A18, A21, T22, A23, F41, K43, D47, S48, V50, D77, M91, M93, E96, S107, E111, A118, P126, K130, I157, N158, A172, A180, L183, 5186, A194, A206, R203, 5207, Y216, M235, F247, S249, S252, and/or G262 relative to the amino acid sequence set forth in SEQ ID NO: 1.

In some embodiments, the recombinant esterase (e.g., a PETase) (or a functional fragment, functional variant, or domain thereof) comprises one or more (e.g., 1, 2, 3, 4, 5, 6, 7, 8, 9, 10, 11, 12, 13, 14, 15, 16, 17, 18, 19, 21, 22, 23, 24, 25, 26, 27, 28, 29, 30, 31, 32, 33, 34, 35, 36) of the following amino acid substitutions S11T, T14E, A18N, A21E, A21V, T22A, A23S, F41L, K43S, D47G, S48G, V50I, D77E, M91I, M93I, E96N, S107N, E111A, A118T, P126A, K130M, I157L, N158K, A172S, A180T, L183I, S186Q, A194S, R203N, A206D, A206G, S207T, Y216N, M235K, F247N, S249T, S249I, S249T, S252D, or G262S relative to the amino acid sequence set forth in SEQ ID NO: 1.

In some embodiments, the recombinant esterase (e.g., a PETase) (or a functional fragment, functional variant, or domain thereof) comprises (a) an amino acid variation (e.g., substitution) at amino acid position Y216; (b) an amino acid variation (e.g., substitution) at amino acid position A172; (c) an amino acid variation (e.g., substitution) at amino acid position F247; (d) an amino acid variation (e.g., substitution) at amino acid position N158; (e) an amino acid variation (e.g., substitution) at amino acid position R203; (f) an amino acid variation (e.g., substitution) at amino acid position S48; (g) an amino acid variation (e.g., substitution) at amino acid position S429; (h) an amino acid variation (e.g., substitution) at amino acid position M235; (i) an amino acid variation (e.g., substitution) at each of amino acid positions A18, A21, M91, M93, E96, K130, 1157, A180, A194, A206, S207, and S249; (j) an amino acid variation (e.g., substitution) at each of amino acid positions S11, V50, D77, A118, A180, S186, and S249; or (k) an amino acid variation (e.g., substitution) at each of amino acid positions T14, A18, A21, T22, A23, F41, K43, D47, D77, M93, E96, S107, E111, P126, A172, A180, L183, A206, Y216, M235, S249, S252, and G262; each amino acid variation (e.g., substitution) in (a)-(k) is relative to the amino acid sequence set forth in SEQ ID NO: 1.

In some embodiments, the recombinant esterase (e.g., a PETase) (or a functional fragment, functional variant, or domain thereof) comprises (a) a Y216N amino acid substitution; (b) an A172S amino acid substitution; (c) a F247N amino acid substitution; (d) an N158K amino acid substitution; (e) a R203N amino acid substitution; (f) a S48G amino acid substitution; (g) a S429I amino acid substitution; (h) a M235K amino acid substitution; (i) an A18N, A21V, M91I, M93I, E96N, K130M, I157L, A180T, A194S, A206G, S207T, and a S249T amino acid substitution; (j) a S11T, V50I, D77E, A118T, A180T, S186Q, and a S249T amino acid substitution; or (k) a T14E, A18N, A21E, T22A, A23S, F41L, K43S, D47G, D77E, M93I, E96N, S107N, E111A, P126A, A172S, A180T, L183I, A206D, Y216N, M235K, S249T, S252D, and a G262S amino acid substitution; each amino acid variation (e.g., substitution) in (a)-(k) is relative to the amino acid sequence set forth in SEQ ID NO: 1.

In some embodiments, the esterase has PETase activity. In some embodiments, esterase is a PETase. In some embodiments, the esterase exhibits enhanced esterase activity relative to a reference esterase (e.g., PETase) (e.g., a reference esterase (e.g., PETase) comprising the amino acid sequence set forth in SEQ ID NO: 1) (e.g., at specified conditions) (e.g., as determined as described in Example 2). In some embodiments, the esterase exhibits enhanced PETase activity relative to a reference PETase (e.g., a reference PETase comprising the amino acid sequence set forth in SEQ ID NO: 1) (e.g., at specified conditions) (e.g., as determined as described in Example 2).

In one aspect, provided herein are fusion proteins comprising an esterase (e.g., a PETase) described herein operably connected to a heterologous protein. In some embodiments, the heterologous protein is a cellular export signal peptide or a protein tag.

In one aspect, provided herein are conjugates comprising an esterase (e.g., a PETase) described herein operably connected to a heterologous moiety.

In one aspect, provided herein are nucleic acid molecules encoding an esterase (e.g., a PETase) described herein, a fusion protein described herein, or a conjugate described herein. In some embodiments, the nucleic acid molecule is a DNA, RNA, or DNA/RNA hybrid molecule.

In one aspect, provided herein are vectors comprising a nucleic acid molecule described herein. In some embodiments, the vector is a viral vector or a non-viral vector (e.g., plasmid, minicircle).

In one aspect, provided herein are cells (e.g., host cells) comprising an esterase (e.g., a PETase) described herein, a fusion protein described herein, a conjugate described herein, a nucleic acid molecule described herein, or a vector described herein. In some embodiments, the host cell is a prokaryotic cell or a eukaryotic cell. In some embodiments, the host cell is a bacterial, mammalian, yeast, fungal, insect, or plant cell. In some embodiments, the host cell is a bacterial cell.

In one aspect, provided herein are cells (e.g., host cells) genetically modified to express an esterase (e.g., a PETase) described herein, a fusion protein described herein, or a conjugate described herein. In some embodiments, the host cell is a prokaryotic cell or a eukaryotic cell. In some embodiments, the host cell is a bacterial, mammalian, yeast, fungal, insect, or plant cell. In some embodiments, the host cell is a bacterial cell.

In one aspect, provided herein is a population of cells (e.g., host cells) comprising cells (e.g., host cells) described herein.

In one aspect, provided herein are compositions comprising an esterase (e.g., a PETase) described herein, a fusion protein described herein, a conjugate described herein, a nucleic acid molecule described herein, a vector described herein, a cell described herein, or a population of cells described herein. In some embodiments, the composition is a liquid composition or a dry composition (e.g., a powder composition, a lyophilizate composition). In some embodiments, the composition further comprises an additional enzyme (e.g., a hydrolase (e.g., an esterase (e.g., a PETase); a NMHETase).

In one aspect, provided herein are reaction mixtures comprising (a) an esterase (e.g., a PETase) described herein, a fusion protein described herein, a conjugate described herein, a nucleic acid molecule described herein, a vector described herein, a cell described herein, a population of cells described herein, or a composition described herein; and (b) a terephthalic acid ester (e.g., a mono-, di-, or poly-terephthalic acid ester) (e.g., polyethylene terephthalate (PET)), a polyester (e.g., PET), or a plastic product comprising a polyester (e.g., PET).

In one aspect, provided herein are reactor vessels comprising an esterase (e.g., a PETase) described herein, a fusion protein described herein, a conjugate described herein, a nucleic acid molecule described herein, a vector described herein, a cell described herein, a population of cells described herein, a composition described herein, or a reaction mixture described herein.

In one aspect, provided herein are kits comprising an esterase (e.g., a PETase) described herein, a fusion protein described herein, a conjugate described herein, a nucleic acid molecule described herein, a vector described herein, a cell described herein, a population of cells described herein, a composition described herein, or a reaction mixture described herein; and optionally instructions for using any one or more of the foregoing.

In one aspect, provided herein are methods (e.g., an in vitro method) of producing an esterase (e.g., a PETase) described herein, the method comprising: a) introducing a nucleic acid molecule encoding an esterase (e.g., PETase) described here (e.g., a nucleic acid molecule described herein, or a vector described herein) into a host cell; b) culturing the host cell under conditions and for an amount of time suitable to allow expression of the esterase (e.g., PETase); and optionally c) isolating and/or purifying the esterase (e.g., PETase).

In one aspect, provided herein are methods of hydrolyzing a terephthalic acid ester, the method comprising contacting a terephthalic acid ester with an esterase (e.g., a PETase) described herein, a fusion protein described herein, a conjugate described herein, a nucleic acid molecule described herein, a vector described herein, a cell described herein, a population of cells described herein, or a composition described herein, to thereby hydrolyze the terephthalic acid ester.

In some embodiments, the terephthalic acid ester is a mono-terephthalic acid ester, a di-terephthalic acid ester, or a poly-terephthalic acid ester. In some embodiments, the terephthalic acid ester is comprised in a polyethylene terephthalate (PET).

In some embodiments, the terephthalic acid ester is comprised in a polyester. In some embodiments, the polyester comprises polyethylene terephthalate (PET), polylactic acid (PLA), polytrimethylene terephthalate (PTT), polybutylene terephthalate (PBT), polyethylene isosorbide terephthalate (PEIT), polyethylene terephthalate (PET), polyhydroxyalkanoate (PHA), polybutylene succinate (PBS), polybutylene succinate adipate (PBSA), polybutylene adipate terephthalate (PBAT), polyethylene furanoate (PEF), polycapro lactone (PCL), polyethylene adipate (PEA), poly(glycolic acid) (PGA), poly(lactic-co-glycolic acid) (PLGA), or any combination of any of the foregoing. In some embodiments, the polyester comprises PET.

In some embodiments, the hydrolysis results in the production of any one or more of terephthalic acid (TPA), mono-(2-hydroxyethyl) terephthalate (MHET), bis-(2-hydroxyethyl) terephthalate (BHET), and/or and ethylene glycol (EG) (or any combination thereof).

In one aspect, provided herein are methods of hydrolyzing a polyethylene terephthalate (PET), the method comprising contacting a PET with a terephthalic acid ester with an esterase (e.g., a PETase) described herein, a fusion protein described herein, a conjugate described herein, a nucleic acid molecule described herein, a vector described herein, a cell described herein, a population of cells described herein, or a composition described herein, to thereby hydrolyze the PET.

In some embodiments, the PET is comprised in a polyester. In some embodiments, the polyester additionally comprises any one or more of polylactic acid (PLA), polytrimethylene terephthalate (PTT), polybutylene terephthalate (PBT), polyethylene isosorbide terephthalate (PEIT), polyethylene terephthalate (PET), polyhydroxyalkanoate (PHA), polybutylene succinate (PBS), polybutylene succinate adipate (PBSA), polybutylene adipate terephthalate (PBAT), polyethylene furanoate (PEF), polycapro lactone (PCL), polyethylene adipate (PEA), poly(glycolic acid) (PGA), poly(lactic-co-glycolic acid) (PLGA), or any combination of any of the foregoing.

In some embodiments, the hydrolysis results in the production of any one or more of terephthalic acid (TPA), mono-(2-hydroxyethyl) terephthalate (MHET), bis-(2-hydroxyethyl) terephthalate (BHET), and/or and ethylene glycol (EG) (or any combination thereof).

In one aspect, provided herein are methods of hydrolyzing a polyester, the method comprising contacting a polyester with a terephthalic acid ester with an esterase (e.g., a PETase) described herein, a fusion protein described herein, a conjugate described herein, a nucleic acid molecule described herein, a vector described herein, a cell described herein, a population of cells described herein, or a composition described herein, to thereby hydrolyze the polyester.

In some embodiments, the polyester comprises polyethylene terephthalate (PET), polylactic acid (PLA), polytrimethylene terephthalate (PTT), polybutylene terephthalate (PBT), polyethylene isosorbide terephthalate (PEIT), polyethylene terephthalate (PET), polyhydroxyalkanoate (PHA), polybutylene succinate (PBS), polybutylene succinate adipate (PBSA), polybutylene adipate terephthalate (PBAT), polyethylene furanoate (PEF), polycapro lactone (PCL), polyethylene adipate (PEA), poly(glycolic acid) (PGA), poly(lactic-co-glycolic acid) (PLGA), or any combination of any of the foregoing. In some embodiments, the polyester comprises PET.

In some embodiments, the hydrolysis results in the production of any one or more of terephthalic acid (TPA), mono-(2-hydroxyethyl) terephthalate (MHET), bis-(2-hydroxyethyl) terephthalate (BHET), and/or and ethylene glycol (EG) (or any combination thereof).

In one aspect, provided herein are methods of degrading a plastic product comprising a polyester, the method comprising contacting the plastic product with a terephthalic acid ester with an esterase (e.g., a PETase) described herein, a fusion protein described herein, a conjugate described herein, a nucleic acid molecule described herein, a vector described herein, a cell described herein, a population of cells described herein, or a composition described herein, to thereby degrade the plastic product.

In some embodiments, the polyester comprises polyethylene terephthalate (PET), polyethylene terephthalate (PET), polylactic acid (PLA), polytrimethylene terephthalate (PTT), polybutylene terephthalate (PBT), polyethylene isosorbide terephthalate (PEIT), polyhydroxyalkanoate (PHA), polybutylene succinate (PBS), polybutylene succinate adipate (PBSA), polybutylene adipate terephthalate (PBAT), polyethylene furanoate (PEF), polycapro lactone (PCL), polyethylene adipate (PEA), poly(glycolic acid) (PGA), poly(lactic-co-glycolic acid) (PLGA), or any combination of any of the foregoing. In some embodiments, the polyester comprises PET.

In some embodiments, the degradation results in the production of any one or more of terephthalic acid (TPA), mono-(2-hydroxyethyl) terephthalate (MHET), bis-(2-hydroxyethyl) terephthalate (BHET), and/or and ethylene glycol (EG) (or any combination thereof). In some embodiments, the plastic product comprises a textile, container (e.g., bag, bottle), film, or powder.

In one aspect, provided herein are methods of recycling a plastic product comprising a polyester, the method comprising contacting the plastic product with a terephthalic acid ester with an esterase (e.g., a PETase) described herein, a fusion protein described herein, a conjugate described herein, a nucleic acid molecule described herein, a vector described herein, a cell described herein, a population of cells described herein, or a composition described herein, to thereby recycle the plastic product.

In some embodiments, the polyester comprises polyethylene terephthalate (PET), polyethylene terephthalate (PET), polylactic acid (PLA), polytrimethylene terephthalate (PTT), polybutylene terephthalate (PBT), polyethylene isosorbide terephthalate (PEIT), polyhydroxyalkanoate (PHA), polybutylene succinate (PBS), polybutylene succinate adipate (PBSA), polybutylene adipate terephthalate (PBAT), polyethylene furanoate (PEF), polycapro lactone (PCL), polyethylene adipate (PEA), poly(glycolic acid) (PGA), poly(lactic-co-glycolic acid) (PLGA), or any combination of any of the foregoing. In some embodiments, the polyester comprises PET.

In some embodiments, the degradation results in the production of any one or more of terephthalic acid (TPA), mono-(2-hydroxyethyl) terephthalate (MHET), bis-(2-hydroxyethyl) terephthalate (BHET), and/or and ethylene glycol (EG) (or any combination thereof).

In some embodiments, the plastic product comprises a textile, container (e.g., bag, bottle), film, or powder.

In some embodiments, any one of the foregoing methods (i.e., any one of the foregoing aspects) further comprises recovering, isolating, and/or purifying the NMET, BHET, TPA, and/or EG, or any combination thereof.

In one aspect, provided herein are compositions comprising the recovered NMET, BHET, TPA, and/or EG, or any combination thereof.

In one aspect, provided herein are methods of producing PET, the method comprising utilizing the NMET, BHET, TPA, and/or EG, or any combination thereof, produced by any one or more of the methods described herein or recovered, isolated, and/or purified by according to any one or more of the methods described herein. In some embodiments, the method comprises a polycondensation process.

In one aspect, provided herein are compositions comprising the PET made by any one or more of the methods described herein. In some embodiments, the composition comprises a polyester. In some embodiments, the composition is a plastic product. In some embodiments, the plastic product is a textile, container (e.g., bag, bottle), film, or powder.

In one aspect, provided herein are methods of producing a plastic product, the method comprising utilizing the NMET, BHET, TPA, and/or EG, or any combination thereof, produced by any one or more of the methods described herein or recovered, isolated, and/or purified by any one or more of the methods described herein. In some embodiments, the method comprises a polycondensation process. In some embodiments, the plastic product comprises PET. In some embodiments, the plastic product comprises a textile, container (e.g., bag, bottle), film, or powder.

In one aspect, provided herein are plastic products made by any one or more of the methods described herein.

### 4. DETAILED DESCRIPTION

Polyethylene terephthalate (PET) based plastics are one of the most commonly used and a major contributor to global plastics waste pollution. PET is commonly used for example in e.g., textiles, packing materials, plastic bottles, food jars, and films. Importantly, PET does not readily decompose in nature, contributing, at least in part, to its major role in environmental plastic pollution. Approaches to deal with the problem of plastic waste products have typically included incineration, disposal in landfills, and mechanical disintegration. However, these approaches also have a significant environmental impact. Enzymatic and chemical methods of degradation have been considered but are relatively inefficient with the enzymes tested or require the use of relatively hazardous chemicals, respectively. As such, a diverse set of PET degrading enzymes (i.e., PETases) are useful to provide e.g., efficient, and environmentally friendly methods of degrading plastic waste. The inventors have, inter alia, discovered novel esterases (e.g., PETases). As such, the esterases (e.g., PETases) described herein can be used degrade plastic products comprising e.g., polyesters (e.g., PET). Accordingly, the current disclosure provides, inter alia, esterases (e.g., PETases) capable of hydrolyzing PET; as well as nucleic acids encoding the same, cell and compositions comprising the same.

### 4.1 Definitions

The section headings used herein are for organizational purposes and do not limit the subject matter described.

Unless defined otherwise, all technical and scientific terms used herein have the same meaning as is understood by one of skill in the art to which the claimed subject matter belongs. It is to be understood that the general and detailed descriptions are exemplary and explanatory and are not restrictive of claimed subject matter.

In this application, the use of the singular includes the plural unless stated otherwise. For example, as used in the disclosure, the singular forms “a,” “an,” and “the” include plural referents unless the context dictates otherwise. Furthermore, use of the term “including” as well as other forms, such as “include,” “includes,” and “included,” is not limiting.

It is understood that aspects and embodiments described herein with “comprising” language, also otherwise include analogous aspects and embodiments described in terms of “consisting of” and “consisting essentially of.”

The term “and/or” is to be taken as specific disclosure of each of the two specified features or components with or without the other. Thus, “and/or” as used in a phrase such as “A and/or B” herein is intended to include “A and B,” “A or B,” “A” (alone), and “B” (alone). Likewise, “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As described herein, concentration ranges, percentage ranges, ratio ranges or integer ranges are understood to include the value of any integer within the recited range and, when appropriate, fractions thereof (such as one tenth and one hundredth of an integer), unless otherwise indicated.

The term “about” refers to a value or composition that is within an acceptable error range for the particular value or composition as understood and/or determined by one of ordinary skill in the art, which will depend in part on how the value or composition is measured or determined, i.e., limitations of the measurement system. When particular values or compositions are provided in the disclosure, unless otherwise stated, the meaning of “about” is understood to be within an acceptable error range for that particular value or composition.

Where proteins are described herein, it is understood that polynucleotides (e.g., RNA or DNA nucleic acid molecules) encoding the proteins are also provided herein.

Where proteins, nucleic acid molecules, vectors, carriers, etc. are described herein, it is understood that isolated forms of the proteins, nucleic acid molecules, vectors, carriers, etc. are also provided herein.

Where proteins, nucleic acid molecules, etc. are described herein, it is understood that recombinant forms of the proteins, nucleic acid molecules, etc. are also provided herein.

Where proteins or sets of proteins are described herein, it is understood that both proteins comprising the primary structure are provided herein as well as proteins folded into their three-dimensional structure (i.e., tertiary or quaternary structure) are provided herein.

As used herein, the terms “agent” and “moiety” are used interchangeably herein and refer to any macro or micro molecule that can be operably connected to another macro or micro molecule (e.g., a protein (e.g., an esterase (or a functional fragment, functional variant, or domain thereof)) or a nucleic acid molecule encoding the protein (e.g., esterase)). Exemplary moieties include, but are not limited small molecules, proteins, polynucleotides (e.g., DNA, RNA), carbohydrates, lipids, synthetic polymers (e.g., polymers of PEG).

As used herein, the term “cell” or “cells” refers to both the subject cell or cells and progeny thereof. The progeny of a parent cell that may not be identical to the parent cell due to e.g., mutations that occur during replication.

As used herein, the term “cutinase” refers to an enzyme that is capable of catalyzing the cleavage (e.g., hydrolysis) or modification (e.g., transesterification) of an ester bond of cutin.

The terms “DNA” and “polydeoxyribonucleotide” are used interchangeably and refer to macromolecules including multiple deoxyribonucleotides that are polymerized via phosphodiester bonds. Deoxyribonucleotides are nucleotides in which the sugar is deoxyribose.

As used herein, the term “domain” refers to a structure of a biomolecule (e.g., a protein, nucleic acid (e.g., DNA, RNA)) molecule) that contributes to a specified function of the biomolecule (e.g., a protein, nucleic acid (e.g., DNA, RNA)). A domain may comprise a contiguous region (e.g., a contiguous sequence) or distinct non-contiguous regions (e.g., non-contiguous sequences) of a biomolecule.

As used herein, the term “esterase” refers to an enzyme that can catalyze any one or more of, ester formation, ester cleavage (e.g., the hydrolysis of an ester bond to produce an acid and an alcohol), transesterification, and/or interesterification. Esterase activities, include, for example, but are not limited to, PETase activity, lipase activity, cutinase activity, etc. Esterases can have more than on enzymatic activity. For example, an esterase can exhibit PETase, lipase, and cutinase activity, acyltransferase activity (e.g., for transesterification), or any combination thereof.

As used herein, the term “functional fragment” in reference to a protein refers to a fragment of a reference protein that retains at least one particular function. Not all functions of the reference protein need be retained by a functional fragment of the protein. In some instances, one or more functions are selectively reduced or eliminated. In some embodiments, the reference protein is a wild type protein. In some embodiments, the functional fragment comprises one or more domains (e.g., 1, 2, 3, or more) of the reference protein.

As used herein, the term “functional variant” in reference to a protein refers to a protein that comprises at least one but not more than 20%, not more than 15%, not more than 12%, no more than 10%, no more than 8% amino acid variation (e.g., substitution, deletion, addition) compared to the amino acid sequence of a reference protein, wherein the protein retains at least one particular function of the reference protein. Not all functions of the reference protein (e.g., wild type) need be retained by the functional variant of the protein. In some instances, one or more functions are selectively altered, reduced or eliminated. In some embodiments, the reference protein is a wild type protein. In some embodiments, the functional variant comprises one or more domains (e.g., 1, 2, 3, or more) of the reference protein.

As used herein, the term “functional fragment or variant thereof” and the like with reference to an agent (e.g., a protein) should be understood to include (a) functional variants, (b) functional fragments, and (c) functional fragments and functional variants.

As used herein, the term “fuse” and grammatical equivalents thereof refers to the operable connection of at least a first polypeptide to a second polypeptide, wherein the first and second polypeptides are not naturally found operably connected together. For example, the first and second polypeptides are derived from different proteins and/or are from different organisms. The term fuse encompasses both a direct connection of the at least two polypeptides through a peptide bond, and the indirect connection through a linker (e.g., a peptide linker).

As used herein, the term “fusion protein” and grammatical equivalents thereof refer to a protein that comprises at least one polypeptide operably connected to another polypeptide, wherein the first and second polypeptides are not naturally found operably connected together. For example, the first and second polypeptides of the fusion protein are each derived from different proteins and/or are from heterologous organisms. For the sake of clarity, it will be understood that neither the first nor second polypeptide is required to be a full-length protein (e.g., a full-length naturally occurring protein). For example, the first and/or second polypeptide can comprise or consist of fragments (e.g., functional fragments or domains of full-length proteins (e.g., engineered, naturally occurring). The at least two polypeptides of the fusion protein can be directly operably connected through a peptide bond; or can be indirectly operably connected through a linker (e.g., a peptide linker). Thus, the term fusion polypeptide encompasses embodiments, wherein Polypeptide A is directly operably connected to Polypeptide B through a peptide bond (Polypeptide A-Polypeptide B), and embodiments, wherein Polypeptide A is operably connected to Polypeptide B through a peptide linker (Polypeptide A-peptide linker-Polypeptide B).

As used herein, the term “heterologous,” when used to describe a first element in reference to a second element means that the first element and second element do not exist in nature disposed as described. For example, a protein comprising a “heterologous moiety” means a protein that is joined to a moiety (e.g., small molecule, protein, polynucleotide, carbohydrate, lipid, synthetic polymer (e.g., polymers of PEG), etc.) that is not joined to the protein in nature.

It is clear from the disclosure, but for the sake of clarity, it is to be understood that the use of the term “heterologous protein” (e.g., any heterologous protein described herein) includes the full-length protein, as well as less than the full-length protein, including, e.g., functional fragments, functional variants, and domains of the full-length protein.

As used herein, the term “hydrolase” refers to an enzyme that is capable of catalyzing the hydrolysis of chemical bonds, including ester bonds.

As used herein, the term “isolated” with reference to a biomolecule (e.g., a protein or polynucleotide) refers to a biomolecule (e.g., a protein or polynucleotide) that is substantially free of other cellular components with which it is associated in the natural state.

As used herein, the term “lipase” refers to an enzyme capable of catalyzing the cleavage (e.g., hydrolysis) or modification (e.g., transesterification) of an ester bond of a triglyceride. In some embodiments, the enzyme is capable of catalyzing the cleavage of ester bonds in triglycerides via hydrolysis. In some embodiments, the triglyceride is broken down into free fatty acid and glycerol.

The terms “nucleic acid molecule” and “polynucleotide” are used interchangeably herein and refer to a polymer of DNA or RNA. The nucleic acid molecule can be single-stranded or double-stranded; contain natural, non-natural, or altered nucleotides; and contain a natural, non-natural, or altered internucleotide linkage, including a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan appreciates that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA polynucleotides encoded by a DNA identified by a particular sequence identification number may also comprise the corresponding RNA (e.g., mRNA) sequence encoded by the DNA, where each thymidine (T) of the DNA sequence is substituted with uracil (U).

As used herein, the term “operably connected” refers to the linkage of two moieties in a functional relationship. For example, a polypeptide is operably connected to another polypeptide when they are linked (either directly or indirectly via a peptide linker) such that both polypeptides are functional (e.g., an in-frame fusion protein comprising an esterase described herein). Or for example, a transcription regulatory polynucleotide e.g., a promoter, enhancer, or other expression control element operably linked to a polynucleotide that encodes a protein to affect the transcription of the polynucleotide that encodes the protein. The term “operably connected” also refers to the conjugation of a moiety to e.g., a polynucleotide or polypeptide (e.g., the conjugation of a PEG polymer to a protein).

Determination of “percent identity” between two sequences (e.g., protein (amino acid sequences) or polynucleotide (nucleic acid sequences)), as used herein, can be accomplished using a mathematical algorithm. For example, a specific, non-limiting example of an algorithm utilized for the comparison of two sequences is described in Karlin S & Altschul S F (1990) PNAS 87: 2264-2268, modified as in Karlin S & Altschul S F (1993) PNAS 90: 5873-5877, each of which is herein incorporated by reference in its entirety. Such algorithm(s) is incorporated into the NBLAST and XBLAST programs of Altschul S F et al., (1990) J Mol Biol 215: 403, which is incorporated herein by reference in its entirety. BLAST nucleotide searches are performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. For gapped alignment comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform searches which detect distant relationships between molecules (Id.). When utilizing BLAST, Gapped BLAST, and PSI Blast programs, default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is described in Myers and Miller, 1988, CABIOS 4:11-17, which is herein incorporated by reference in its entirety. Such an algorithm is incorporated in the ALIGN program (version 2.0) and is a part of the GCG sequence alignment software package. When comparing amino acid sequences with the ALIGN program, a PAM120 weight residue table, a gap length penalty of 12, and a gap penalty of 4 can be used. Percent identity between two sequences can be determined using techniques similar to those described above, with or without allowing gaps. In calculating percent identity, typically only exact matches are counted.

As used herein, the term “PETase” refers to an enzyme that is capable of catalyzing the breakdown and/or modification of polyethylene terephthalate (PET). In some embodiments, the PETase exhibits hydrolase activity (e.g., carboxyl ester hydrolase activity) and/or peptidase activity. In some embodiments, the PETase is capable of catalyzing the hydrolysis and/or transesterification of PET. In some embodiments, the PETase catalyzes the hydrolysis of PET plastic to at least mono-2-hydroxyethyl terephthalate (MHET). Additional products may also be produced, including e.g., TPA and EG. A PETase can also exhibit other activity (e.g., other enzymatic activity) distinct from the PETase activity. For example, a PETase described herein may also exhibit cutinase activity, lipase activity, acyltransferase activity, etc. or any combination thereof.

As used herein, the term “plurality” means 2 or more (e.g., 3 or more, 4 or more, 5 or more, 6 or more, 7 or more, 9 or more, or 10 or more).

As used herein, the term “polymer” typically refers to a chemical compound or a mixture of compounds whose structure is made up of multiple products (repeat units) linked by covalent chemical bonds. Within the context of the present disclosure, the term polymer includes natural or synthetic polymers, constituted of a single type of repeat unit (i.e., homopolymers) or of a mixture of different repeat units (i.e., copolymers or heteropolymers).

As used herein, the terms “polyester containing material”, “polyester containing product” and the like are to be understood as refers to a product, such as plastic product, comprising at least one polyester in crystalline, semi-crystalline or totally amorphous form. The polyester containing material may refer to any item made from at least one plastic material, such as plastic sheet, tube, rod, profile, shape, film, massive block, fiber, textiles, etc., which contains at least one polyester, and possibly other substances or additives, such as plasticizers, mineral or organic fillers. In an embodiment, the polyester containing material is a textile or fabric comprising at least one polyester containing fiber. In another embodiment, the polyester containing material is a plastic compound, or plastic formulation, in a molten or solid state, suitable for making a plastic product. Suitable polyesters will be familiar to persons skilled in the art, illustrative examples of which include polylactic acid (PLA), polyethylene terephthalate (PET), polytrimethylene terephthalate (PTT), polybutylene terephthalate (PBT), polyethylene isosorbide terephthalate (PEIT), polyhydroxyalkanoate (PHA), polybutylene succinate (PBS), polybutylene succinate adipate (PBSA), polybutylene adipate terephthalate (PBAT), polyethylene furanoate (PEF), polycaprolactone (PCL), and poly(ethylene adipate) (PEA). Thus, in an embodiment, the polyester is selected from the group consisting of polylactic acid (PLA), polyethylene terephthalate (PET), polytrimethylene terephthalate (PTT), polybutylene terephthalate (PBT), polyethylene isosorbide terephthalate (PEIT), polyhydroxyalkanoate (PHA), polybutylene succinate (PBS), polybutylene succinate adipate (PBSA), polybutylene adipate terephthalate (PBAT), polyethylene furanoate (PEF), polycaprolactone (PCL), poly(ethylene adipate) (PEA) and combinations of any of the foregoing.

As used herein, the terms “protein” and “polypeptide” refer to a polymer of at least 2 (e.g., at least 5) amino acids linked by a peptide bond. The term “polypeptide” does not denote a specific length of the polymer chain of amino acids. It is common in the art to refer to shorter polymers of amino acids (e.g., approximately 2-50 amino acids) as peptides; and to refer to longer polymers of amino acids (e.g., approximately over 50 amino acids) as polypeptides. However, the terms “peptide” and “polypeptide” and “protein” are used interchangeably herein. In some embodiments, a protein is folded into its three-dimensional structure. Where proteins are contemplated herein, it should be understood that proteins comprising the primary structure are provided herein as well as proteins folded into their three-dimensional structure (i.e., tertiary or quaternary structure) are provided herein.

As used herein, the term “recycling” generally refers to the conversion of a material generally not considered suitable for practical use into a reusable material. For example, post-consumer waste, including e.g., plastic bottles, plastic film, textiles, may generally not be considered suitable for practical use. In addition to post-consumer waste, manufacturing waste and excess stock may also be considered not suitable for practical use.

The terms “RNA” and “polyribonucleotide” are used interchangeably herein and refer to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleotide linkages, such as a phosphoroamidate linkage or a phosphorothioate linkage, instead of the phosphodiester found between the nucleotides of an unmodified nucleic acid molecule. RNA molecules include, e.g., “translatable RNAs” which refers to any RNA that encodes at least one polypeptide and can be translated to produce the encoded protein in vitro, in vivo, in situ or ex vivo. A translatable RNA may be an mRNA or a circular RNA encoding a polypeptide.

As used herein, the term “signal peptide” or “signal sequence” refers to a sequence that can direct the transport or localization of a protein, such as an esterase, to a certain organelle, cell compartment, or extracellular export. The term encompasses both the signal sequence peptide and the nucleic acid sequence encoding the signal peptide. Thus, references to a signal peptide in the context of a nucleic acid refers to the nucleic acid sequence encoding the signal peptide. Exemplary signal sequences include for example, nuclear localization signal and nuclear export signal.

As used herein, the term terms “variant” or “variation” with reference to a nucleic acid molecule (e.g., a nucleic acid molecule encoding an esterase as described herein), refer to a nucleic acid molecule that comprises at least one substitution, inversion, addition, or deletion of nucleotide compared to a reference nucleic acid molecule. As used herein, the term “variant” or “variation” with reference to a protein refers to a peptide or protein (e.g., esterases described herein) that comprises at least one substitution, inversion, addition, or deletion of an amino acid residue compared to a reference protein.

### 4.2 Esterases

Provided herein are, inter alia, esterases (e.g., PETases) (and functional fragments, functional variants, and domains thereof), useful in, inter alia, methods of degrading a polyester containing products (e.g., plastic products).

As described above, esterases are a class of enzymes that can catalyze any one or more of, ester formation, ester cleavage (e.g., the hydrolysis of an ester bond to produce an acid and an alcohol), transesterification, and/or interesterification. Esterases can have more than on enzymatic activity. For example, an esterase can exhibit PETase activity, lipase activity, cutinase activity, acyltransferase activity (e.g., for transesterification), etc., or any combination thereof. In some embodiments, the esterase is capable of hydrolyzing an ester bond into an acid and an alcohol. In some embodiments, the esterase is capable of mediating transesterification. In some embodiments, the esterase exhibit lipase activity. In some embodiments, the esterase exhibits cutinase activity. In some embodiments, the esterase exhibits acyltransferase activity. In some embodiments, the esterase is capable of mediating ester synthesis, capable of mediating ester hydrolysis, capable of mediating transesterification, exhibits lipase activity, exhibits cutinase activity, acyltransferase activity, or any combination of any of the foregoing (e.g., any 1, 2, 3, 4, 5, or 6 of the foregoing).

As described above, PETases are a class of enzymes capable of catalyzing the breakdown and/or modification of polyethylene terephthalate (PET). In some embodiments, the PETase exhibits carboxyl ester hydrolase activity and/or peptidase activity. In some embodiments, the PETase is capable of catalyzing the hydrolysis and/or transesterification of PET. In some embodiments, the PETase catalyzes the hydrolysis of PET plastic to at least mono-2-hydroxyethyl terephthalate (MHET). Additional products may also be produced, including e.g., TPA and EG. A PETase can also exhibit other activity (e.g., other enzymatic activity) distinct from the PETase activity. For example, a PETase described herein may also exhibit cutinase activity, lipase activity, or any combination thereof. In some embodiments, the PETase is capable of hydrolyzing polyethylene terephthalate (PET) into one or more products (e.g., monomers, subunit e.g., mono-2-hydroxyethyl terephthalate (MHET). NMHET can be further broken down into products (e.g., monomers), e.g., terephthalic acid (TPA) and ethylene glycol (EG). The amino acid sequence of an exemplary reference esterase (e.g., PETase) is set forth in Table 1.

TABLE 1
The Amino Acid Sequence of Exemplary Reference
Esterase (e.g., PETase).

| | | SEQ ID |
|---|---|---|
| Description | Amino Acid Sequence | NO |
| Reference | FSAPGPSAPCSNCTRGPAPTATALKASTGPFSTAKFSVSGFLKGFGDSTVYY | 1 |
| Esterase 1 | PTNTTGKMGAIAVIPGYLSYESSIDWWGPRLASHGFVVMTMNTETIYDQPDS | |
| | RASQLSEALDYLIAQSGSSSSPLYNKIDSTRLGAIGWSMGGGGSLKLSTQRS | |
| | INAIIPQAPWYSGSNAFNTIKTPALILACSADTVAPVAQHASPFYNRIPAST | |
| | PKAFLEIYGGSHFCANSGYPNEDLLGMYGIAWMKRFIDFDSRYSQFLCGPNH | |
| | EGDLAISEYRQNCPY | |

In some embodiments, the esterase (e.g., PETase) is non-naturally occurring. In some embodiments, the amino acid sequence of the esterase (e.g., PETase) comprises one or more amino acid variation (e.g., substitution) relative to the amino acid sequence of a reference esterase (e.g., PETase). In some embodiments, the amino acid sequence of the esterase (e.g., PETase) comprises one or more amino acid variation (e.g., substitution) relative to the amino acid sequence of the reference esterase (e.g., PETase) set forth in SEQ ID NO: 1.

In some embodiments, the esterase (e.g., PETase) is non-naturally occurring. In some embodiments, the amino acid sequence of the esterase (e.g., PETase) comprises one or more amino acid variation (e.g., substitution) relative to the amino acid sequence of a reference esterase (e.g., PETase). In some embodiments, the amino acid sequence of the esterase (e.g., PETase) comprises one or more amino acid variation (e.g., substitution) relative to the amino acid sequence of the reference esterase (e.g., PETase) set forth in Table 1 or set forth in any one of SEQ ID NO: 1.

In some embodiments, the esterase (e.g., PETase) is non-naturally occurring. In some embodiments, the amino acid sequence of the esterase (e.g., PETase) comprises one or more amino acid variation (e.g., substitution) relative to the amino acid sequence of a reference esterase (e.g., PETase). In some embodiments, the amino acid sequence of the esterase (e.g., PETase) comprises one or more amino acid variation (e.g., substitution) relative to the amino acid sequence of the reference esterase (e.g., PETase) set forth in SEQ ID NO: 1.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises one or more amino acid variation (e.g., substitution or deletion).

In some embodiments, the amino acid sequence of the esterase (e.g., PETase) (or the functional fragment, functional variant, or domain thereof) is less than 100% (e.g., less than 99%, 98%, 97%, 96%, 95%, 94%, 93%, 92%, 91%, or 90%) identical to the amino acid sequence set forth in SEQ ID NO: 1. In some embodiments, the amino acid sequence of the esterase (e.g., PETase) (or the functional fragment, functional variant, or domain thereof) comprises at least one (e.g., 2, 3, 4, 5, 6, 7, 8, 9, 10, 11, 12, 13, 14, 15, 16, 17, 18, 19, 20, 21, 22, 23, 24, 25) amino acid variation (e.g., substitution) relative to the amino acid sequence set forth in SEQ ID NO: 1.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises one or more amino acid variation (e.g., substitution). In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 1 and comprises an amino acid variation (e.g., substitution) at one or more (e.g., 1, 2, 3, 4, 5, 6, 7, 8, 9, 10, 11, 12, 13, 14, 15, 16, 17, 18, 19, 21, 22, 23, 24, 25, 26, 27, 28, 29, 30, 31, 32, 33, 34, 35, 36) of amino acid positions S11, T14, A18, A21, T22, A23, F41, K43, D47, S48, V50, D77, M91, M93, E96, S107, E111, A118, P126, K130, 1157, N158, A172, A180, L183, S186, A194, A206, R203, S207, Y216, M235, F247, S249, S252, and/or G262.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises one or more (e.g., 1, 2, 3, 4, 5, 6, 7, 8, 9, 10, 11, 12, 13, 14, 15, 16, 17, 18, 19, 21, 22, 23, 24, 25, 26, 27, 28, 29, 30, 31, 32, 33, 34, 35, 36) of the following amino acid substitutions S11T, T14E, A18N, A21E, A21V, T22A, A23S, F41L, K43S, D47G, S48G, V50I, D77E, M91I, M93I, E96N, S107N, E111A, A118T, P126A, K130M, I157L, N158K, A172S, A180T, L183I, S186Q, A194S, R203N, A206D, A206G, S207T, Y216N, M235K, F247N, S249T, S249I, S249T, S252D, and/or G262S.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises an amino acid variation (e.g., substitution) at amino acid position Y216.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises an amino acid variation (e.g., substitution) at amino acid position A172.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises an amino acid variation (e.g., substitution) at amino acid position F247.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises an amino acid variation (e.g., substitution) at amino acid position N158.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises an amino acid variation (e.g., substitution) at amino acid position R203.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises an amino acid variation (e.g., substitution) at amino acid position S48.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises an amino acid variation (e.g., substitution) at amino acid position S429.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises an amino acid variation (e.g., substitution) at amino acid position M235.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises an amino acid variation (e.g., substitution) at each of amino acid positions A18, A21, M91, M93, E96, K130, 1157, A180, A194, A206, S207, and S249.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises an amino acid variation (e.g., substitution) at each of amino acid positions 511, V50, D77, A118, A180, S186, and S249.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises an amino acid variation (e.g., substitution) at each of amino acid positions T14, A18, A21, T22, A23, F41, K43, D47, D77, M93, E96, S107, E111, P126, A172, A180, L183, A206, Y216, M235, S249, S252, and G262.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises a Y216N amino acid substitution.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises an A172S amino acid substitution.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises a F247N amino acid substitution.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises an N158K amino acid substitution.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises a R203N amino acid substitution.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises a S48G amino acid substitution.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises a S429I amino acid substitution.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises a M235K amino acid substitution.

In some embodiments, the esterase (e.g., PETase) comprises an amino acid sequence at least 80%, 81%, 82% 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises each of the following amino acid substitutions A18N, A21V, M91I, M93I, E96N, K130M, I157L, A180T, A194S, A206G, S207T, and S249T.

In some embodiments, the esterase (e.g., PETase) comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises each of the following amino acid substitutions S11T, V50I, D77E, A1118T, A180T, S186Q, and S249T.

In some embodiments, the esterase (e.g., PETase) comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises each of the following amino acid substitutions T14E, A18N, A21E, T22A, A23S, F41L, K43S, D47G, D77E, M93I, E96N, S107N, E111A, P126A, A172S, A180T, L183I, A2061D, Y216N, M235K, S249T, S252D3, and G262S.

The amino acid sequence of exemplary esterases (e.g., PETases) (e.g., non-naturally occurring) of the of the disclosure is set forth in Table 2 and in SEQ ID NOS: 2-12.

TABLE 2
The Amino Acid Sequence of Esterases (e.g., PETases).

| | | SEQ ID |
|---|---|---|
| Description | Amino Acid Sequence | NO |
| Esterase-1 | FSAPGPSAPCSNCTRGPAPTATALKASTGPFSTAKFSVSGFLKGFGDSTVYY | 2 |
| | PTNTTGKMGAIAVIPGYLSYESSIDWWGPRLASHGFVVMTMNTETIYDQPDS | |
| | RASQLSEALDYLIAQSGSSSSPLYNKIDSTRLGAIGWSMGGGGSLKLSTQRS | |
| | INAIIPQAPWYSGSNAFNTIKTPALILACSADTVAPVAQHASPFYNRIPAST | |
| | PKAFLEINGGSHFCANSGYPNEDLLGMYGIAWMKRFIDFDSRYSQFLCGPNH | |
| | EGDLAISEYRQNCPY | |
| Esterase-2 | FSAPGPSAPCSNCTRGPAPTATALKASTGPFSTAKFSVSGFLKGFGDSTVYY | 3 |
| | PTNTTGKMGAIAVIPGYLSYESSIDWWGPRLASHGFVVMTMNTETIYDQPDS | |
| | RASQLSEALDYLIAQSGSSSSPLYNKIDSTRLGAIGWSMGGGGSLKLSTQRS | |
| | INAIIPQAPWYSGSNSFNTIKTPALILACSADTVAPVAQHASPFYNRIPAST | |
| | PKAFLEIYGGSHFCANSGYPNEDLLGMYGIAWMKRFIDFDSRYSQFLCGPNH | |
| | EGDLAISEYRQNCPY | |
| Esterase-3 | FSAPGPSAPCSNCTRGPAPTATALKASTGPFSTAKFSVSGFLKGFGDSTVYY | 4 |
| | PTNTTGKMGAIAVIPGYLSYESSIDWWGPRLASHGFVVMTMNTETIYDQPDS | |
| | RASQLSEALDYLIAQSGSSSSPLYNKIDSTRLGAIGWSMGGGGSLKLSTQRS | |
| | INAIIPQAPWYSGSNAFNTIKTPALILACSADTVAPVAQHASPFYNRIPAST | |
| | PKAFLEIYGGSHFCANSGYPNEDLLGMYGIAWMKRFIDNDSRYSQFLCGPNH | |
| | EGDLAISEYRQNCPY | |
| Esterase-4 | FSAPGPSAPCSNCTRGPAPTATALKASTGPFSTAKFSVSGFLKGFGDSTVYY | 5 |
| | PTNTTGKMGAIAVIPGYLSYESSIDWWGPRLASHGFVVMTMNTETIYDQPDS | |
| | RASQLSEALDYLIAQSGSSSSPLYNKIDSTRLGAIGWSMGGGGSLKLSTQRS | |
| | IKAIIPQAPWYSGSNAFNTIKTPALILACSADTVAPVAQHASPFYNRIPAST | |
| | PKAFLEIYGGSHFCANSGYPNEDLLGMYGIAWMKRFIDFDSRYSQFLCGPNH | |
| | EGDLAISEYRQNCPY | |
| Esterase-5 | FSAPGPSAPCSNCTRGPAPTATALKASTGPFSTAKFSVSGFLKGFGDSTVYY | 6 |
| | PTNTTGKMGAIAVIPGYLSYESSIDWWGPRLASHGFVVMTMNTETIYDQPDS | |
| | RASQLSEALDYLIAQSGSSSSPLYNKIDSTRLGAIGWSMGGGGSLKLSTQRS | |
| | INAIIPQAPWYSGSNAFNTIKTPALILACSADTVAPVAQHASPFYNNIPAST | |
| | PKAFLEIYGGSHFCANSGYPNEDLLGMYGIAWMKRFIDFDSRYSQFLCGPNH | |
| | EGDLAISEYRQNCPY | |
| Esterase-6 | FSAPGPSAPCSNCTRGPAPTATALKASTGPFSTAKFSVSGFLKGFGDGTVYY | 7 |
| | PTNTTGKMGAIAVIPGYLSYESSIDWWGPRLASHGFVVMTMNTETIYDQPDS | |
| | RASQLSEALDYLIAQSGSSSSPLYNKIDSTRLGAIGWSMGGGGSLKLSTQRS | |
| | INAIIPQAPWYSGSNAFNTIKTPALILACSADTVAPVAQHASPFYNRIPAST | |
| | PKAFLEIYGGSHFCANSGYPNEDLLGMYGIAWMKRFIDFDSRYSQFLCGPNH | |
| | EGDLAISEYRQNCPY | |
| Esterase-7 | FSAPGPSAPCSNCTRGPNPTVTALKASTGPFSTAKFSVSGFLKGFGDSTVYY | 8 |
| | PTNTTGKMGAIAVIPGYLSYESSIDWWGPRLASHGEVVITINTNTIYDQPDS | |
| | RASQLSEALDYLIAQSGSSSSPLYNMIDSTRLGAIGWSMGGGGSLKLSTQRS | |
| | LNAIIPQAPWYSGSNAFNTIKTPTLILACSADTVAPVSQHASPFYNRIPGTT | |
| | PKAFLEIYGGSHFCANSGYPNEDLLGMYGIAWMKRFIDFDTRYSQFLCGPNH | |
| | EGDLAISEYRQNCPY | |
| Esterase-8 | FSAPGPSAPCTNCTRGPAPTATALKASTGPFSTAKFSVSGFLKGFGDSTIYY | 9 |
| | PTNTTGKMGAIAVIPGYLSYESSIEWWGPRLASHGFVVMTMNTETIYDQPDS | |
| | RASQLSEALDYLITQSGSSSSPLYNKIDSTRLGAIGWSMGGGGSLKLSTQRS | |
| | INAIIPQAPWYSGSNAFNTIKTPTLILACQADTVAPVAQHASPFYNRIPAST | |
| | PKAFLEIYGGSHFCANSGYPNEDLLGMYGIAWMKRFIDFDTRYSQFLCGPNH | |
| | EGDLAISEYRQNCPY | |
| Esterase-9 | FSAPGPSAPCSNCERGPNPTEASLKASTGPFSTAKFSVSGLLSGFGGSTVYY | 10 |
| | PTNTTGKMGAIAVIPGYLSYESSIEWWGPRLASHGFVVMTINTNTIYDQPDS | |
| | RANQLSAALDYLIAQSGSSSSALYNKIDSTRLGAIGWSMGGGGSLKLSTQRS | |
| | INAIIPQAPWYSGSNSFNTIKTPTLIIACSADTVAPVAQHASPFYNRIPDST | |
| | PKAFLEINGGSHFCANSGYPNEDLLGKYGIAWMKRFIDFDTRYDQFLCGPNH | |
| | ESDLAISEYRQNCPY | |
| Esterase-10 | FSAPGPSAPCSNCTRGPAPTATALKASTGPFSTAKFSVSGFLKGFGDSTVYY | 11 |
| | PTNTTGKMGAIAVIPGYLSYESSIDWWGPRLASHGFVVMTMNTETIYDQPDS | |
| | RASQLSEALDYLIAQSGSSSSPLYNKIDSTRLGAIGWSMGGGGSLKLSTQRS | |
| | INAIIPQAPWYSGSNAFNTIKTPALILACSADTVAPVAQHASPFYNRIPAST | |
| | PKAFLEIYGGSHFCANSGYPNEDLLGMYGIAWMKRFIDFDIRYSQFLCGPNH | |
| | EGDLAISEYRQNCPY | |
| Esterase-11 | FSAPGPSAPCSNCTRGPAPTATALKASTGPFSTAKFSVSGELKGFGDSTVYY | 12 |
| | PTNTTGKMGAIAVIPGYLSYESSIDWWGPRLASHGFVVMTMNTETIYDQPDS | |
| | RASQLSEALDYLIAQSGSSSSPLYNKIDSTRLGAIGWSMGGGGSLKLSTQRS | |
| | INAIIPQAPWYSGSNAFNTIKTPALILACSADTVAPVAQHASPFYNRIPAST | |
| | PKAFLEIYGGSHFCANSGYPNEDLLGKYGIAWMKRFIDFDSRYSQFLCGPNH | |
| | EGDLAISEYRQNCPY | |

In some embodiments, the amino acid sequence of the esterase (e.g., PETase) (or the functional fragment, functional variant, or domain thereof) comprises or consists of an amino acid sequence at least about 80%, 81%, 82%, 83%, 84%, 85%, 86%, 87%, 88%, 89%, 90%, 91%, 92%, 93%, 94%, 95%, 96%, 97%, 98%, 99% or 100% identical to the amino acid sequence of any polypeptide set forth in Table 2 or set forth in any one of SEQ ID NOS: 2-12. In some embodiments, the amino acid sequence of the esterase (e.g., PETase) (or the functional fragment, functional variant, or domain thereof) comprises or consists of an amino acid sequence at least about 85%, 86%, 87%, 88%, 89%, 90%, 91%, 92%, 93%, 94%, 95%, 96%, 97%, 98%, 99%, or 100% identical to the amino acid sequence of any polypeptide set forth in Table 2 or set forth in any one of SEQ ID NOS: 2-12. In some embodiments, the amino acid sequence of the esterase (e.g., PETase) (or the functional fragment, functional variant, or domain thereof) comprises or consists of an amino acid sequence at least about 90%, 91%, 92%, 93%, 94%, 95%, 96%, 97%, 98%, 99%, or 100% identical to the amino acid sequence of any polypeptide set forth in Table 2 or set forth in any one of SEQ ID NOS: 2-12.

In some embodiments, the amino acid sequence of the esterase (e.g., PETase) (or the functional fragment, functional variant, or domain thereof) comprises or consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of a polypeptide set forth in Table 2. In some embodiments, the amino acid sequence of the esterase (e.g., PETase) (or the functional fragment, functional variant, or domain thereof) comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 2. In some embodiments, the amino acid sequence of the esterase (e.g., PETase) (or the functional fragment, functional variant, or domain thereof) comprises or consists of an amino acid sequence at least 90%, 91%, 92%, 93%, 94%, 95%, 96%, 97%, 98%, 99%, or 100% identical to the amino acid sequence of a polypeptide set forth in Table 2.

In some embodiments, the amino acid sequence of the esterase (e.g., PETase) (or the functional fragment, functional variant, or domain thereof) comprises or consists of the amino acid sequence of a polypeptide set forth in Table 2, and further comprises 1 or more but less than 20% (e.g., less than 15%, less than 12%, less than 10%, less than 8%) amino acid variations (e.g., substitutions, additions, deletions, etc.). In some embodiments, the amino acid sequence of the esterase (e.g., PETase) (or the functional fragment, functional variant, or domain thereof) comprises or consists of the amino acid sequence of a polypeptide set forth in Table 2, and further comprises or consists of at least about 1, 2, 3, 4, 5, 6, 7, 8, 9, 10, 20, 30, 40, 50, 60, 70, 80, 90, or 100 amino acid variations (e.g., substitutions, additions, deletions, etc.). In some embodiments, the amino acid sequence of the esterase (e.g., PETase) (or the functional fragment, functional variant, or domain thereof) comprises or consists of the amino acid sequence of a polypeptide set forth in Table 2, and further comprises or consists of about 1, 2, 3, 4, 5, 6, 7, 8, 9, 10, 20, 30, 40, 50, 60, 70, 80, 90, or 100 amino acid variations (e.g., substitutions, additions, deletions, etc.). In some embodiments, the amino acid sequence of the esterase (e.g., PETase) (or the functional fragment, functional variant, or domain thereof) comprises or consists of the amino acid sequence of a polypeptide set forth in Table 2, and further comprises or consists of no more than about 1, 2, 3, 4, 5, 6, 7, 8, 9, 10, 20, 30, 40, 50, 60, 70, 80, 90, or 100 amino acid variations (e.g., substitutions, additions, deletions, etc.). In some embodiments, the amino acid sequence of the esterase (e.g., PETase) (or the functional fragment, functional variant, or domain thereof) comprises or consists of the amino acid sequence of a polypeptide set forth in Table 2, and further comprises or consists of from about 1-100, 1-90, 1-80, 1-70, 1-60, 1-50, 1-40, 1-30, 1-20, 1-10, 1-9, 1-8, 1-7, 1-6, 1-5, 1-4, 1-3, 1-2, 10-100, 10-90, 10-80, 10-70, 10-60, 10-50, 1-40, 10-30, or 10-20 amino acid variations (e.g., substitutions, additions, deletions, etc.).

In some embodiments, the amino acid sequence of the esterase (e.g., PETase) (or the functional fragment, functional variant, or domain thereof) comprises or consists of the amino acid sequence of a polypeptide set forth in Table 2, and further comprises 1 or more but less than 20% (e.g., less than 15%, less than 12%, less than 10%, less than 8%) amino acid substitutions. In some embodiments, the amino acid sequence of the esterase (e.g., PETase) (or the functional fragment, functional variant, or domain thereof) comprises or consists of the amino acid sequence of a polypeptide set forth in Table 2, and further comprises or consists of at least about 1, 2, 3, 4, 5, 6, 7, 8, 9, 10, 20, 30, 40, 50, 60, 70, 80, 90, or 100 amino acid substitutions. In some embodiments, the amino acid sequence of the esterase (e.g., PETase) (or the functional fragment, functional variant, or domain thereof) comprises or consists of the amino acid sequence of a polypeptide set forth in Table 2, and further comprises or consists of about 1, 2, 3, 4, 5, 6, 7, 8, 9, 10, 20, 30, 40, 50, 60, 70, 80, 90, or 100 amino acid substitutions. In some embodiments, the amino acid sequence of the esterase (e.g., PETase) (or the functional fragment, functional variant, or domain thereof) comprises or consists of the amino acid sequence of a polypeptide set forth in Table 2, and further comprises or consists of no more than about 1, 2, 3, 4, 5, 6, 7, 8, 9, 10, 20, 30, 40, 50, 60, 70, 80, 90, or 100 amino acid substitutions. In some embodiments, the amino acid sequence of the esterase (e.g., PETase) (or the functional fragment, functional variant, or domain thereof) comprises or consists of the amino acid sequence of a polypeptide set forth in Table 2, and further comprises or consists of from about 1-100, 1-90, 1-80, 1-70, 1-60, 1-50, 1-40, 1-30, 1-20, 1-10, 1-9, 1-8, 1-7, 1-6, 1-5, 1-4, 1-3, 1-2, 10-100, 10-90, 10-80, 10-70, 10-60, 10-50, 1-40, 10-30, or 10-20 amino acid substitutions.

In some embodiments, the amino acid sequence of the esterase (e.g., PETase) (or the functional fragment, functional variant, or domain thereof) comprises or consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 2-12. In some embodiments, the amino acid sequence of esterase (e.g., PETase) (or the functional fragment, functional variant, or domain thereof) comprises or consists of an amino acid sequence at least 85%, 86%, 87%, 88%, 89%, 90%, 91%, 92%, 93%, 94%, 95%, 96%, 97%, 98%, or 99% identical to the amino acid sequence of any one of SEQ ID NOS: 2-12. In some embodiments, the amino acid sequence of esterase (e.g., PETase) (or the functional fragment, functional variant, or domain thereof) comprises or consists of an amino acid sequence at least 90%, 91%, 92%, 93%, 94%, 95%, 96%, 97%, 98%, 99%, or 100% identical to the amino acid sequence of any one of SEQ ID NOS: 2-12.

In some embodiments, the amino acid sequence of the esterase (e.g., PETase) (or the functional fragment, functional variant, or domain thereof) comprises or consists of the amino acid sequence of any one of SEQ ID NOS: 2-12, and further comprises 1 or more but less than 20% (e.g., less than 15%, less than 12%, less than 10%, less than 8%) amino acid variations (e.g., substitutions, additions, deletions, etc.). In some embodiments, the amino acid sequence of the esterase (e.g., PETase) (or the functional fragment, functional variant, or domain thereof) comprises or consists of the amino acid sequence of any one of SEQ ID NOS: 2-12, and further comprises or consists of at least about 1, 2, 3, 4, 5, 6, 7, 8, 9, 10, 20, 30, 40, 50, 60, 70, 80, 90, or 100 amino acid variations (e.g., substitutions, additions, deletions, etc.). In some embodiments, the amino acid sequence of the esterase (e.g., PETase) (or the functional fragment, functional variant, or domain thereof) comprises or consists of the amino acid sequence of any one of SEQ ID NOS: 2-12, and further comprises or consists of about 1, 2, 3, 4, 5, 6, 7, 8, 9, 10, 20, 30, 40, 50, 60, 70, 80, 90, or 100 amino acid variations (e.g., substitutions, additions, deletions, etc.). In some embodiments, the amino acid sequence of the esterase (e.g., PETase) (or the functional fragment, functional variant, or domain thereof) comprises or consists of the amino acid sequence of any one of SEQ ID NOS: 2-12, and further comprises or consists of no more than about 1, 2, 3, 4, 5, 6, 7, 8, 9, 10, 20, 30, 40, 50, 60, 70, 80, 90, or 100 amino acid variations (e.g., substitutions, additions, deletions, etc.). In some embodiments, the amino acid sequence of the esterase (e.g., PETase) (or the functional fragment, functional variant, or domain thereof) comprises or consists of the amino acid sequence of any one of SEQ ID NOS: 2-12, and further comprises or consists of from about 1-100, 1-90, 1-80, 1-70, 1-60, 1-50, 1-40, 1-30, 1-20, 1-10, 1-9, 1-8, 1-7, 1-6, 1-5, 1-4, 1-3, 1-2, 10-100, 10-90, 10-80, 10-70, 10-60, 10-50, 1-40, 10-30, or 10-20 amino acid variations (e.g., substitutions, additions, deletions, etc.).

In some embodiments, the amino acid sequence of the esterase (e.g., PETase) (or the functional fragment, functional variant, or domain thereof) comprises or consists of the amino acid sequence of any one of SEQ ID NOS: 2-12, and further comprises 1 or more but less than 15% (less than 12%, less than 10%, less than 8%), amino acid substitutions. In some embodiments, the amino acid sequence of the esterase (e.g., PETase) (or the functional fragment, functional variant, or domain thereof) comprises or consists of the amino acid sequence of any one of SEQ ID NOS: 2-12, and further comprises or consists of at least about 1, 2, 3, 4, 5, 6, 7, 8, 9, 10, 20, 30, 40, 50, 60, 70, 80, 90, or 100 amino acid substitutions. In some embodiments, the amino acid sequence of the esterase (e.g., PETase) (or the functional fragment, functional variant, or domain thereof) comprises or consists of the amino acid sequence of any one of SEQ ID NOS: 2-12, and further comprises or consists of about 1, 2, 3, 4, 5, 6, 7, 8, 9, 10, 20, 30, 40, 50, 60, 70, 80, 90, or 100 amino acid substitutions. In some embodiments, the amino acid sequence of the esterase (e.g., PETase) (or the functional fragment, functional variant, or domain thereof) comprises or consists of the amino acid sequence of any one of SEQ ID NOS: 2-12, and further comprises or consists of no more than about 1, 2, 3, 4, 5, 6, 7, 8, 9, 10, 20, 30, 40, 50, 60, 70, 80, 90, or 100 amino acid substitutions. In some embodiments, the amino acid sequence of the esterase (e.g., PETase) (or the functional fragment, functional variant, or domain thereof) comprises or consists of the amino acid sequence of any one of SEQ ID NOS: 2-12, and further comprises or consists of from about 1-100, 1-100, 1-90, 1-80, 1-70, 1-60, 1-50, 1-40, 1-30, 1-20, 1-10, 1-9, 1-8, 1-7, 1-6, 1-5, 1-4, 1-3, 1-2, 10-100, 10-90, 10-80, 10-70, 10-60, 10-50, 1-40, 10-30, or 10-20 amino acid substitutions.

### 4.2.1 Activity of Esterases

The esterases (e.g., PETases) described herein can have multiple functions, have domains of different function, etc. In some embodiments, the proteins described herein have one or more enzymatic activity (e.g., esterase activity, PETase activity, hydrolase activity, acyltransferase activity, cutinase activity, lipase activity, etc.). In some embodiments, the protein described herein (e.g., esterase (e.g., PETase)) has hydrolase activity. In some embodiments, a protein described herein has esterase activity. In some embodiments, a protein described herein (e.g., an esterase) has PETase activity. In some embodiments, the esterase has PETase activity. In some embodiments, a protein described herein (e.g., an esterase) has acyltransferase activity. In some embodiments, a protein described herein (e.g., an esterase) has cutinase activity. In some embodiments, a protein described herein (e.g., an esterase) has lipase activity. In some embodiments, a protein described herein (e.g., an esterase) has transesterification activity.

In some embodiments, the esterase (e.g., PETase) has an altered (e.g., enhanced) activity (e.g., enzymatic activity (e.g., esterase activity, PETase activity, hydrolase activity, acyltransferase activity, cutinase activity, lipase activity, etc.)) relative to a reference esterase (e.g., PETase) (e.g., a reference esterase comprising the amino acid sequence set forth in SEQ ID NO: 1). In some embodiments, the esterase (e.g., PETase) has altered (e.g., enhanced) hydrolase activity relative to a reference esterase (e.g., PETase) (e.g., a reference esterase comprising the amino acid sequence set forth in SEQ ID NO: 1). In some embodiments, the esterase (e.g., PETase) has altered (e.g., enhanced) esterase activity relative to a reference esterase (e.g., PETase) (e.g., a reference esterase comprising the amino acid sequence set forth in SEQ ID NO: 1). In some embodiments, the esterase (e.g., PETase) has altered (e.g., enhanced) PETase activity relative to a reference esterase (e.g., PETase) (e.g., a reference esterase comprising the amino acid sequence set forth in SEQ ID NO: 1). In some embodiments, the esterase (e.g., PETase) has altered (e.g., enhanced) acyltransferase activity relative to a reference esterase (e.g., PETase) (e.g., a reference esterase comprising the amino acid sequence set forth in SEQ ID NO: 1). In some embodiments, the esterase (e.g., PETase) has altered (e.g., enhanced) cutinase activity relative to a reference esterase (e.g., PETase) (e.g., a reference esterase comprising the amino acid sequence set forth in SEQ ID NO: 1). In some embodiments, the esterase (e.g., PETase) has altered (e.g., enhanced) lipase activity relative to a reference esterase (e.g., PETase) (e.g., a reference esterase comprising the amino acid sequence set forth in SEQ ID NO: 1).

In some embodiments, the esterase (e.g., PETase) has an enhanced activity (e.g., esterase activity, hydrolase activity, PETase activity, cutinase activity, lipase activity, acyltransferase activity) relative to a reference esterase comprising the amino acid sequence set forth in SEQ ID NO: 1. In some embodiments, the esterase (e.g., PETase) has an enhanced enzymatic activity (e.g., hydrolase activity, esterase activity, PETase activity) relative to a reference esterase comprising the amino acid sequence set forth in SEQ ID NO: 1. Enhanced enzymatic activity (e.g., hydrolase activity, esterase activity, PETase activity) includes e.g., enhanced catalytic activity and/or broader catalytic activity. In some embodiments, the esterase (e.g., PETase) has enhanced hydrolase activity relative a reference esterase comprising the amino acid sequence set forth in SEQ ID NO: 1. In some embodiments, the esterase (e.g., PETase) has enhanced esterase activity relative a reference esterase comprising the amino acid sequence set forth in SEQ ID NO: 1. In some embodiments, the esterase (e.g., PETase) has enhanced PETase activity relative to a reference esterase comprising the amino acid sequence set forth in SEQ ID NO: 1. In some embodiments, the esterase (e.g., PETase) has enhanced cutinase activity relative to a reference esterase comprising the amino acid sequence set forth in SEQ ID NO: 1. In some embodiments, the esterase (e.g., PETase) has enhanced lipase activity relative to a reference esterase comprising the amino acid sequence set forth in SEQ ID NO: 1. In some embodiments, the esterase (e.g., PETase) has enhanced acyltransferase activity relative to a reference esterase comprising the amino acid sequence set forth in SEQ ID NO: 1.

In some embodiments, the activity (e.g., hydrolase activity, esterase activity, PETase activity) of the esterase (e.g., PETase) is increased by at least 5%, 10%, 15%, 20%, 25%, 30%, 40%, 50%, 60%, 670%, 80%, 90%, 100%, 200%, 300%, 400%, 500%, 600%, 700%, 800%, 900%, or 1000% relative to the activity (e.g., esterase activity, hydrolase activity, PETase activity, cutinase activity, lipase activity, acyltransferase activity) of a reference esterase (e.g., PETase) (e.g., a reference esterase comprising the amino acid sequence set forth in SEQ ID NO: 1). In some embodiments, the activity (e.g., hydrolase activity, esterase activity, PETase activity) of the esterase (e.g., PETase) is increased by at least 1-fold, 2-fold, 5-fold, 10-fold, 20-fold, 30-fold, 40-fold, 50-fold, 60-fold, 70-fold, 80-fold, 90-fold, 100-fold, 500-fold, 1000-fold, 5000-fold, 10000-fold relative to the activity (e.g., esterase activity, hydrolase activity, PETase activity, cutinase activity, lipase activity, acyltransferase activity) of a reference esterase (e.g., PETase) (e.g., a reference esterase comprising the amino acid sequence set forth in SEQ ID NO: 1).

In some embodiments, the esterase (e.g., PETase) has enzymatic activity (e.g., esterase activity, hydrolase activity, PETase activity, cutinase activity, lipase activity, acyltransferase activity) at a range of temperatures. In some embodiments, the esterase (e.g., PETase) has enzymatic activity (e.g., esterase activity, hydrolase activity, PETase activity, cutinase activity, lipase activity, acyltransferase activity) at from about 10 °C to about 80 °C, 20 °C to about 80 °C, 30 °C to about 80 °C, 40 °C to about 80 °C, 50 °C to about 80 °C, 60 °C to about 80 °C, or 70 °C to about 80 °C. In some embodiments, the esterase (e.g., PETase) has enzymatic activity (e.g., esterase activity, hydrolase activity, PETase activity, cutinase activity, lipase activity, acyltransferase activity) at about 10 °C, 20 °C, 30 °C, 40 °C, 50 °C, 60 °C, 70 °C, or 80 °C. In some embodiments, the esterase (e.g., PETase) has enzymatic activity (e.g., esterase activity, hydrolase activity, PETase activity, cutinase activity, lipase activity, acyltransferase activity) at a range of pHs. In some embodiments, the esterase (e.g., PETase) has enzymatic activity (e.g., esterase activity, hydrolase activity, PETase activity, cutinase activity, lipase activity, acyltransferase activity) at a pH from about 5 to about 11, 6 to about 11, 7 to about 11, 5 to about 10, 6 to about 10, or 7 to about 10.

Suitable methods of determining or measuring the activity (e.g., enzymatic activity (e.g., esterase activity, hydrolase activity, PETase activity, cutinase activity, lipase activity, acyltransferase activity) of a protein will be familiar to persons skilled in the art. For example, Example 2 set forth herein describes an exemplary method of determining PETase activity. In some embodiments, the PETase activity of a protein described herein (e.g., an esterase) is determined as set forth in Example 2. For further example, PETase activity can be determined by UV absorbance assay to measure the amount of product (e.g., IHET, TPA, etc.) produced from using amorphous PET as a substrate. Another method useful for determining or measuring the PETase activity of a protein is by measuring the amount of IHET and/or TPA produced using Analytical High Performance Liquid Chromatography (HPLC). For further example, methods are described in Palm et al. (2019, Nat. Comm., 10:1717); Sagong et al. (2020, ACS Catal. 10:4805); Yoshida et al. (2016, Science, 352(6278): 1196); and Zhong-Johnson, E. Z. L., Voigt, C. A., & Sinskey, A. J. (2021). An absorbance method for analysis of enzymatic degradation kinetics of poly(ethylene terephthalate) films. Scientific reports, 11(1), 928. https://doi.org/10.1038/s41598-020-79031-5, the entire contents of each of which are incorporated herein by reference for all purposes.

### 4.3 Esterase Fusion Proteins and Conjugates

In some embodiments, an esterase (e.g., PETase) (or a functional fragment, functional variant, or domain thereof) described herein (or a nucleic acid molecule encoding an esterase (e.g., PETase) (or a functional fragment, functional variant, or domain thereof) described herein) is operably connected to a heterologous moiety (e.g., a heterologous protein (e.g., or a functional fragment, functional variant, or domain thereof)). As such, further provided herein are, inter alia, fusion proteins comprising an esterase (e.g., PETase) (e.g., described herein) (or a functional fragment, functional variant, or domain thereof) and one or more heterologous protein (or a functional fragment, functional variant, or domain thereof). Further provided herein are, inter alia, conjugates comprising an esterase (e.g., PETase) (e.g., described herein) (or a functional fragment, functional variant, or domain thereof) (or a nucleic acid molecule encoding an esterase (e.g., PETase) (or a functional fragment, functional variant, or domain thereof) described herein) and one or more heterologous moiety.

Heterologous moieties include, but are not limited to, proteins, peptides, small molecules, nucleic acid molecules (e.g., DNA, RNA, DNA/RNA hybrid molecules), carbohydrates, lipids, and polymers (e.g., synthetic polymers).

In some embodiments, the esterase (e.g., PETase) (or a functional fragment, functional variant, or domain thereof) is operably connected to at least 1, 2, 3, 4, 5, 6, 7, 8, 9, or 10, or more heterologous moieties. In some embodiments, the esterase (e.g., PETase) (or a functional fragment, functional variant, or domain thereof) is operably connected to 1, 2, 3, 4, 5, 6, 7, 8, 9, or 10, but no more than 10 heterologous moieties. In some embodiments, the esterase (e.g., PETase) (or a functional fragment, functional variant, or domain thereof) is operably connected to no more than 1, 2, 3, 4, 5, 6, 7, 8, 9, or 10, heterologous moieties. In some embodiments, the esterase (e.g., PETase) (or the functional fragment or functional variant thereof) is operably connected to from about 1-10, 1-9, 1-8, 1-7, 1-6, 1-5, 1-4, 1-3, or 1-2 heterologous moieties. In some embodiments, the esterase (e.g., PETase) (or a functional fragment, functional variant, or domain thereof) is operably connected to about 1, 2, 3, 4, 5, 6, 7, 8, 9, or 10, heterologous moieties.

### 4.3.1 Heterologous Proteins

In some embodiments, the heterologous moiety is a protein. As such, as described above, provided herein are fusion proteins comprising an esterase (e.g., PETase) (e.g., described herein) (or a functional fragment, functional variant, or domain thereof) and one or more heterologous protein. It is clear from the disclosure, but for the sake of clarity, it is to be understood that the use of the term “heterologous protein” (e.g., any heterologous protein described herein) includes a full-length protein, as well as e.g., functional fragments, functional variants, and domains of the full-length protein.

In some embodiments, the fusion protein comprises more than one heterologous protein. In some embodiments, the fusion protein comprises a plurality of heterologous proteins. In some embodiments, the esterase (e.g., PETase) (or a functional fragment, functional variant, or domain thereof) is operably connected to at least 1, 2, 3, 4, 5, 6, 7, 8, 9, or 10, or more heterologous proteins. In some embodiments, the esterase (e.g., PETase) (or a functional fragment, functional variant, or domain thereof) is operably connected to at least 1, 2, 3, 4, 5, 6, 7, 8, 9, or 10, but no more than 10 heterologous proteins. In some embodiments, the esterase (e.g., PETase) (or a functional fragment, functional variant, or domain thereof) is operably connected to no more than 1, 2, 3, 4, 5, 6, 7, 8, 9, or 10, heterologous proteins. In some embodiments, the esterase (e.g., PETase) (or a functional fragment, functional variant, or domain thereof) is operably connected to from about 1-10, 1-9, 1-8, 1-7, 1-6, 1-5, 1-4, 1-3, or 1-2 heterologous proteins (or a functional fragment, functional variant, or domain thereof). In some embodiments, the esterase (e.g., PETase) (or a functional fragment, functional variant, or domain thereof) is operably connected to about 1, 2, 3, 4, 5, 6, 7, 8, 9, or 10, heterologous proteins.

Exemplary heterologous proteins include, but are not limited to, detectable proteins (e.g., fluorescent proteins, protein tags (e.g., FLAG tags, HIS tags, HA tags), reporter genes); protein tags (e.g., FLAG tags, HIS tags, HA tags); immobilization proteins (e.g., FLAG tags, HIS tags, HA tags); cellular export signal peptides; and enzymes.

In some embodiments, the heterologous protein is a protein tag. In some embodiments, the heterologous protein is a protein tag suitable for use in purification of the fusion protein. In some embodiments, the heterologous protein is a protein tag suitable for immobilization of the fusion protein on a substrate (e.g., as described herein).

In some embodiments, the heterologous protein is a cellular export signal peptide. In some embodiments, the heterologous protein is an N-terminal cellular export signal peptide.

In some embodiments, the heterologous protein is an enzyme. In some embodiments, the heterologous protein exhibits enzymatic activity (e.g., MHETase activity).

### 4.3.2 Linkers

As described herein, a heterologous moiety (e.g., heterologous protein) can be directly operably connected or indirectly operably connected to an esterase (e.g., PETase) (e.g., described herein). In some embodiments, the heterologous protein is directly operably connected to an esterase (e.g., PETase) (e.g., described herein). In some embodiments, a heterologous polypeptide is directly operably connected to an esterase (e.g., PETase) (e.g., described herein) via a peptide bond. In some embodiments, a heterologous protein is indirectly operably connected to an esterase (e.g., PETase) (e.g., described herein). In some embodiments, a heterologous protein is indirectly operably connected to an esterase (e.g., PETase) (e.g., described herein) via a linker.

In some embodiments, a heterologous protein is indirectly operably connected to an esterase (e.g., PETase) (e.g., described herein) via a peptide linker. In some embodiments, a peptide linker is one or any combination of a cleavable linker, a non-cleavable linker, a flexible linker, a rigid linker, a helical linker, and/or a non-helical linker. In some embodiments, a peptide linker comprises from or from about 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, or 5-10 amino acid residues. In some embodiments, the peptide linker comprises at least about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, a linker comprises or consists of about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the linker comprises or consists of no more than about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the amino acid sequence of the peptide linker comprises or consists of glycine, serine, or both glycine and serine amino acid residues. In some embodiments, an amino acid sequence of the peptide linker comprises or consists of glycine, serine, and proline amino acid residues.

The amino acid sequence of exemplary peptide linkers is provided in Table 3.

TABLE 3
The Amino Acid Sequence of Exemplary
Peptide Linker.

| Descrip- | | SEQ ID |
|---|---|---|
| tion | Amino Acid Sequence | NO |
| A | GGG | 13 |
| B | GGGG | 14 |
| C | GGGGG | 15 |
| D | GGGGGG | 16 |
| E | GGGGGGG | 17 |
| F | GGGGGGGG | 18 |
| G | GSS | 19 |
| H | GSSGSS | 20 |
| I | GSSGSSGSS | 21 |
| J | GSSGSSGSSGSS | 22 |
| K | GSSGSSGSSGSSGSS | 23 |
| L | GSSGSSGSSGSSGSSGSS | 24 |
| M | GGS | 25 |
| N | GGSGGS | 26 |
| O | GGSGGSGGS | 27 |
| P | GGSGGSGGSGGS | 28 |
| Q | GGSGGSGGSGGSGGS | 29 |
| R | GGSGGSGGSGGSGGSGGS | 30 |
| S | GGGGS | 31 |
| T | GGGGSGGGGS | 32 |
| U | GGGGSGGGGSGGGGS | 33 |
| V | GGGGSGGGGSGGGGSGGGGSGGGGS | 34 |
| W | GGGGSGGGGSGGGGSGGGGSGGGGSGGGGS | 35 |
| X | GGSGGG | 36 |
| Y | GGGGGS | 37 |
| Z | GGSGSS | 38 |
| A-1 | GSSGGS | 39 |
| B-1 | GSS | 40 |
| C-1 | GSSGSS | 41 |
| D-1 | GSSGSSGSS | 42 |
| E-1 | GSSGSSGSSGSS | 43 |
| F-1 | GSSGSSGSSGSSGSS | 44 |
| G-1 | GSSGSSGSSGSSGSSGSS | 45 |
| H-1 | GGGGSS | 46 |
| I-1 | GGSGGGGSS | 47 |
| J-1 | GGSGSSGGG | 48 |
| K-1 | GGGGGSGSS | 49 |
| L-1 | GGGGSSGGS | 50 |
| M-1 | GSSGGSGGG | 51 |
| N-1 | GSSGGGGGS | 52 |
| O-1 | EAAAK | 53 |
| P-1 | EAAAKEAAAK | 54 |
| Q-1 | EAAAKEAAAKEAAAK | 55 |
| R-1 | EAAAKEAAAKEAAAKEAAAK | 56 |
| S-1 | EAAAKEAAAKEAAAKEAAAKEAAAK | 57 |
| T-1 | EAAAKEAAAKEAAAKEAAAKEAAAKEAAAK | 58 |
| U-1 | GGSGGGEAAAK | 59 |
| V-1 | GGSEAAAKGGG | 60 |
| W-1 | GGGGGSEAAAK | 61 |
| X-1 | GGGEAAAKGGS | 62 |
| Y-1 | EAAAKGGSGGG | 63 |
| Z-1 | EAAAKGGGGGS | 64 |
| A-2 | PAP | 65 |
| B-2 | PAPAP | 66 |
| C-2 | PAPAPAP | 67 |
| D-2 | PAPAPAPAP | 68 |
| E-2 | PAPAPAPAPAP | 69 |
| F-2 | PAPAPAPAPAPAP | 70 |

In some embodiments, an amino acid sequence of the peptide linker comprises or consists of the amino acid sequence of any one of the linkers set forth in Table 3. In some embodiments, the amino acid sequence of the peptide linker comprises or consists of the amino acid sequence of any one of the linkers set forth in Table 3, and further comprises 1 or more but less than 15% (less than 12%, less than 10%, less than 8%), amino acid variations (e.g., amino acid substitutions, deletions, or additions). In some embodiments, the amino acid sequence of the peptide linker comprises or consists of the amino acid sequence of any one of the linkers set forth in Table 3, comprising 1, 2, or 3 amino acid variations (e.g., substitutions, deletions, additions). In some embodiments, the amino acid sequence of the peptide linker comprises or consists of the amino acid sequence of any one of the linkers set forth in Table 3, and further comprises 1 or more but less than 15% (less than 12%, less than 10%, less than 8%), amino acid substitutions. In some embodiments, the amino acid sequence of the peptide linker comprises or consists of the amino acid sequence of any one of the linkers set forth in Table 3, comprising 1, 2, or 3 amino acid substitutions.

In some embodiments, an amino acid sequence of the peptide linker comprises or consists of the amino acid sequence of any one of SEQ ID NOS: 13-70. In some embodiments, the amino acid sequence of the peptide linker comprises or consists of the amino acid sequence of any one of SEQ ID NOS: 13-70, and further comprises 1 or more but less than 15% (less than 12%, less than 10%, less than 8%), amino acid variations (e.g., amino acid substitutions, deletions, or additions). In some embodiments, the amino acid sequence of the peptide linker comprises or consists of the amino acid sequence of any one of SEQ ID NOS: 13-70, comprising 1, 2, or 3 amino acid variations (e.g., substitutions, deletions, additions). In some embodiments, the amino acid sequence of the peptide linker comprises or consists of the amino acid sequence of any one of SEQ ID NOS: 13-70, and further comprises 1 or more but less than 15% (less than 12%, less than 10%, less than 8%), amino acid substitutions. In some embodiments, the amino acid sequence of the peptide linker comprises or consists of the amino acid sequence of any one of SEQ ID NOS: 13-70, comprising 1, 2, or 3 amino acid substitutions.

### 4.3.3 Orientation

The heterologous moiety (or moieties) (e.g., heterologous protein(s)) and the esterase (e.g., PETase) (e.g., described herein) (or a functional fragment, functional variant, or domain thereof) can be arranged in any configuration or order as long as the esterase (e.g., PETase) (e.g., described herein) (or a functional fragment, functional variant, or domain thereof) maintains the ability to mediate its function and in the embodiments wherein the heterologous moiety (e.g., heterologous protein) has a specific function, the heterologous moiety (e.g., heterologous protein) can mediate its function.

In some embodiments, the heterologous moiety (e.g., heterologous protein) is operably connected to the N-terminus, C-terminus, or internally between the N-terminus and the C-terminus of the esterase (e.g., PETase) (or a functional fragment, functional variant, or domain thereof). In some embodiments, a heterologous moiety (e.g., heterologous protein) is operably connected to the C-terminus of the esterase (e.g., PETase) (or the functional fragment, functional variant, or domain thereof). In some embodiments, a heterologous moiety (e.g., heterologous protein) is operably connected to the N-terminus of the esterase (e.g., PETase) (or the functional fragment, functional variant, or domain thereof) and a heterologous moiety (e.g., heterologous protein) is operably connected to the C-terminus of the esterase (e.g., PETase) (or the functional fragment, functional variant, or domain thereof).

### 4.4 Methods of Making Esterases and Fusion Proteins

Proteins described herein (e.g., esterases (e.g., PETases), fusion proteins, and conjugates) may be produced using standard methods known in the art. For example, each may be produced by recombinant technology in host cells (e.g., insect cells, mammalian cells, bacteria) that have been transfected or transduced with a nucleic acid expression vector (e.g., plasmid, viral vector (e.g., a baculoviral expression vector)) encoding the protein (e.g., the esterase, fusion protein, etc.). Such general methods are common knowledge in the art. The expression vector typically contains an expression cassette that includes nucleic acid sequences capable of bringing about expression of the nucleic acid molecule encoding the protein of interest (e.g., the esterase (e.g., PETase), fusion protein, etc.), such as promoter(s), enhancer(s), polyadenylation signals, and the like. The person of ordinary skill in the art is aware that various promoter and enhancer elements can be used to obtain expression of a nucleic acid molecule in a host cell. For example, promoters can be constitutive or regulated, and can be obtained from various sources, e.g., viruses, prokaryotic or eukaryotic sources, or artificially designed. Post transfection or transduction, host cells containing the expression vector encoding the protein of interest are cultured under conditions conducive to expression of the nucleic acid molecule encoding the protein of interest (e.g., the esterase (e.g., PETase), fusion protein, etc.). Culture media is available from various vendors, and a suitable medium can be routinely chosen for a host cell to express a protein of interest. Host cells can be adherent or suspension cultures, and a person of ordinary skill in the art can optimize culture methods for specific host cells selected. For example, suspension cells can be cultured in, for example, bioreactors in e.g., a batch process or a fed-batch process. The produced protein may be isolated from the cell cultures, by, for example, column chromatography in either flow-flow through or bind-and-elute modes. Examples include, but are not limited to, ion exchange resins and affinity resins, such as lentil lectin Sepharose, and mixed mode cation exchange-hydrophobic interaction columns (CEX-HIC). The protein may be concentrated, buffer exchanged by ultrafiltration, and the retentate from the ultrafiltration may be filtered through an appropriate filter, e.g., a 0.22 μm filter. See, e.g., Hacker, David (Ed.), Recombinant Protein Expression in Mammalian Cells: Methods and Protocols (Methods in Molecular Biology), Humana Press (2018). See also U.S. Pat. No. 5,762,939, the entire contents of each of which is incorporated by reference herein for all purposes. Proteins described herein (e.g., esterases (e.g., PETases), fusion proteins, and protein conjugates) may be produced synthetically.

The disclosure provides, inter alia, methods of making a protein described herein (e.g., an esterase (e.g., PETase) (or a functional fragment, functional variant, or domain thereof), a fusion protein, etc.) comprising (a) introducing a nucleic acid molecule encoding the protein (e.g., the esterase (e.g., PETase) (or the functional fragment, functional variant, or domain thereof), the fusion protein etc.) into a host cell; (b) culturing the host cell (e.g., under conditions and for a period of time sufficient to allow expression of the protein (e.g., the esterase (e.g., PETase) (or the functional fragment, functional variant, or domain thereof), the fusion protein etc.); and optionally isolating the protein (e.g., the esterase (e.g., PETase) (or the functional fragment, functional variant, or domain thereof), the fusion protein etc.) from the culture medium.

### 4.5 Nucleic Acid Molecules

In one aspect, provided herein are polynucleotides (e.g., DNA, RNA) encoding any protein described herein (including, e.g., an esterase (e.g., a PETase) described herein (see, e.g., §), a fusion protein described herein (see, e.g., §), a conjugate described herein (see, e.g., § Error! Reference source not found.)). In some embodiments, the polynucleotide is a DNA polynucleotide or an RNA polynucleotide. In some embodiments, the polynucleotide is a translatable RNA polynucleotide. In some embodiments, the polynucleotide is an mRNA polynucleotide. In some embodiments, the polynucleotide is a circular RNA polynucleotide. In some embodiments, the polynucleotide is a translatable circular RNA polynucleotide.

In some embodiments, the polynucleotide is codon optimized. Codon optimization may be used to match codon frequencies in target and host organisms to ensure proper folding; bias guanosine (G) and/or cytosine content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g., glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

### 4.6 Vectors

In one aspect, provided herein are vectors comprising a polynucleotide (e.g., DNA, RNA) described herein (e.g., a polynucleotide described in §). In some embodiments, the vector is a viral vector. In some embodiments, the vector is a non-viral vector (e.g., a plasmid, a minicircle).

### 4.6.1 Non-Viral Vectors

In some embodiments, the vector is a non-viral vector. In some embodiments, the vector is a minicircle. In some embodiments, the vector is a plasmid. A person of ordinary skill in the art is aware of suitable plasmids for expression of the DNA of interest. For example, plasmid DNA may be generated to allow efficient production of the encoded esterases in cell lines, e.g., in insect cell lines, for example using vectors as described in WO2009150222A2 and as defined in PCT claims 1 to 33, the disclosure relating to claim 1 to 33 of WO2009150222A2 the entire contents of which is incorporated by reference herein for all purposes.

### 4.6.2 Viral Vectors

In some embodiments, the nucleic acid molecules (e.g., DNA or RNA) encoding an immunogenic peptide or protein described herein are contained in a viral vector. Thus, also provided herein are viral vectors comprising the nucleic acid molecules encoding an immunogenic peptide or protein described herein. Such vectors can be easily manipulated by methods well known to the ordinary person of skill in the art. The vector used can be any vector that is suitable for cloning nucleic acids that can be used for transcription of the nucleic acid molecule of interest.

Viral vectors include both RNA and DNA based vectors. The vectors can be designed to meet a variety of specifications. For example, viral vectors can be engineered to be capable or incapable of replication in prokaryotic and/or eukaryotic cells. In some embodiments, the vector is replication deficient. In some embodiments, the vector is replication competent. Vectors can be engineered or selected that either will (or will not) integrate in whole or in part into the genome of host cells, resulting (or not (e.g., episomal expression)) in stable host cells comprising the desired nucleic acid in their genome.

Exemplary viral vectors include, but are not limited to, adenovirus vectors, adeno-associated virus vectors, lentivirus vectors, retrovirus vectors, poxvirus vectors, parapoxivirus vectors, vaccinia virus vectors, fowlpox virus vectors, herpes virus vectors, adeno-associated virus vectors, alphavirus vectors, lentivirus vectors, rhabdovirus vectors, measles virus, Newcastle disease virus vectors, picornaviruses vectors, or lymphocytic choriomeningitis virus vectors. In some embodiments, the viral vector is an adenovirus vector, adeno-associated virus vector, lentivirus vector, anellovector (as described, for example, in U.S. Pat. No. 11,446,344, the entire contents of which is incorporated by reference herein for all purposes).

In some embodiments, the vector is an adenoviral vector (e.g., human adenoviral vector, e.g., HAdV or AdHu). In some embodiments, the adenovirus vector has the E1 region deleted, rendering it replication-deficient in human cells. Other regions of the adenovirus such as E3 and E4 may also be deleted. Exemplary adenovirus vectors include, but are not limited to, those described in e.g., WO2005071093 or WQ2006048215, the entire contents of each of which is incorporated by reference herein for all purposes. In some embodiments, the adenovirus-based vector used is a simian adenovirus, thereby avoiding dampening of the immune response after vaccination by pre-existing antibodies to common human entities such as AdHu5. Exemplary, simian adenovirus vectors include AdCh63 (see, e.g., WO2005071093, the entire contents of which is incorporated by reference herein for all purposes) or AdCh68.

Viral vectors can be generated through the use of a packaging/producer cell line (e.g., a mammalian cell line) using standard methods known to the person of ordinary skill in the art. Generally, a nucleic acid construct (e.g., a plasmid) encoding the transgene (e.g., an immunogenic peptide or protein described herein) (along with additional elements e.g., a promoter, inverted terminal repeats (ITRs) flanking the transgene, a plasmid encoding e.g., viral replication and structural proteins, along with one or more helper plasmids a host cell (e.g., a host cell line) are transfected into a host cell line (i.e., the packing/producer cell line). In some instances, depending on the viral vector, a helper plasmid may also be needed that include helper genes from another virus (e.g., in the instance of adeno-associated viral vectors). Eukaryotic expression plasmids are commercially available from a variety of suppliers, for example the plasmid series: pcDNA™ pCR3.1™, pCMV™, pFRT™, pVAX1™, pCI™, Nanoplasmid™, and Pcaggs. The person of ordinary skill in the art is aware of numerous transfection methods and any suitable method of transfection may be employed (e.g., using a biochemical substance as carrier (e.g., lipofectamine), by mechanical means, or by electroporation). The cells are cultured under conditions suitable and for a sufficient time for plasmid expression. The viral particles may be purified from the cell culture medium using standard methods known to the person of ordinary skill in the art. For example, by centrifugation followed by e.g., chromatography or ultrafiltration.

### 4.7 Cells

In one aspect, provided herein are cells (e.g., host cells) (e.g., a population of cells (e.g., a population of host cells)) comprising any one or more of an esterase (e.g., PETase) described herein (see, e.g., §), a fusion protein described herein (see, e.g., §), a conjugate described herein (see, e.g., § Error! Reference source not found.), a polynucleotide described herein (see, e.g., § Error! Reference source not found.), or a vector described herein (see, e.g., § Error! Reference source not found.).

In some embodiments, the cell (e.g., host cell) is genetically modified to express an esterase (e.g., PETase) described herein. The cell (e.g., host cell) can be useful, e.g., in the production (expression) of an esterase (e.g., PETase) described herein.

In some embodiments, the cell is a prokaryotic cell. In some embodiments, the cell is a bacteria cell. In some embodiments, the bacterium is a Gram-positive bacterium. In some embodiments, the bacterium is a Gram-negative bacterium. In some embodiments, the cell is a eukaryotic cell. Exemplary eukaryotic cells include, e.g., yeast, fungal, mammalian, insect, and plant cells. In some embodiments, the cell is mammalian cell. In some embodiments, the cell is an animal cell. In some embodiments, the cell is a yeast cell. In some embodiments, the cell is an insect cell. In some embodiments, the cell is a fungal cell. Suitable host cells will be familiar to persons of ordinary skill in the art, illustrative examples of which include, but are not limited to a recombinant Bacillus, recombinant E. coli, recombinant Pseudomonas, recombinant Aspergillus, recombinant Trichoderma, recombinant Streptomyces, recombinant Saccharomyces, recombinant Pichia, recombinant Thermus or recombinant Yarrowia.

Standard methods known in the art (and described elsewhere herein) can be utilized to deliver any one of the foregoing (e.g., esterase (e.g., PETase) protein, fusion protein, vector, polynucleotide, carrier, etc.) into a cell (e.g., a host cell). Standard methods known in the art can be utilized to culture cells (e.g., host cells) in vitro or ex vivo.

### 4.8 Compositions

In one aspect, provided herein are compositions comprising any one or more of an esterase (e.g., PETase) described herein (see, e.g., §), a fusion protein described herein (see, e.g., §), a conjugate described herein (see, e.g., § Error! Reference source not found.), a polynucleotide described herein (see, e.g., § Error! Reference source not found.), a vector described herein (see, e.g., § Error! Reference source not found.), or a cell (or population of cells) described herein (see, e.g., § Error! Reference source not found.). In some embodiments, the composition comprises an esterase (e.g., PETase) described herein. In some embodiments, the composition comprises a fusion protein described herein. In some embodiments, the composition comprises a conjugate described herein. In some embodiments, the composition comprises a polynucleotide described herein (e.g., encoding an esterase (e.g., PETase) described herein). In some embodiments, the composition comprises a cell or population of cells (e.g., a host cell or population of host cells) described herein.

The composition may be liquid or dry, for instance in the form of a powder. In some embodiments, the composition is a lyophilizate. For instance, the composition may comprise the esterase (e.g., PETase) protein, polynucleotide, vector, cell, etc. and optionally one or more excipient and/or reagent etc. Suitable excipients may include buffers commonly used in biochemistry, agents for adjusting pH, preservatives such as sodium benzoate, sodium sorbate or sodium ascorbate, conservatives, protective or stabilizing agents such as starch, dextrin, arabic gum, salts, sugars e.g., sorbitol, trehalose or lactose, glycerol, polyethyleneglycol, polyethene glycol, polypropylene glycol, propylene glycol, divalent ions such as calcium, sequestering agent such as EDTA, reducing agents (e-g., beta-mercaptoethanol, dithiothreitol, ascorbic acid, tris(2-carboxyethyl)phosphine), amino acids, a carrier such as a solvent or an aqueous solution, and the like.

In some embodiments, the composition comprises an esterase (e.g., PETase) protein described herein (the protein may be present in the composition in an isolated or at least partially purified form). In some embodiments, the esterase (e.g., PETase) described herein is solubilized in an aqueous medium together with one or more excipients, such as excipients that may suitably stabilize or protect the esterase (e.g., PETase) from degradation. For example, the esterase (e.g., PETase) described herein may be solubilized in water and then admixed with excipients such as glycerol, sorbitol, dextrin, starch, glycol such as propanediol, salt, etc. The resulting admixture may then be dried so as to obtain a powder. Methods for drying such mixture are well known to the one skilled in the art and include, without limitation, lyophilization, freeze-drying, spray-drying, supercritical drying, down-draught evaporation, thin-layer evaporation, centrifugal evaporation, conveyer drying, fluidized bed drying, drum drying or any combination thereof.

In some embodiments, the composition comprises an esterase (e.g., PETase) protein described herein in an amount of from about 0.1% to about 99.9%, from about 0.1% to about 50%, from about 0.1% to about 30%, from about 0.1% to about 5% by weight of the total weight of the composition. In some embodiments, the composition comprises an esterase (e.g., PETase) protein described herein in an amount of from about 0.1 to about 5% by weight of the total weight of the composition. In some embodiments, the composition comprises an esterase (e.g., PETase) protein described herein in an amount of from about 0.1 to about 0.2% by weight of the total weight of the composition. The amount of esterase (e.g., PETase) in the composition may be suitably adapted by persons skilled in the art, depending e.g., on the nature and/or amount of a substrate (e.g., the polyester containing material) to be degraded (e.g., hydrolyzed) and/or the presence or absence of any additional proteins (e.g., enzymes) in the composition.

In some embodiments, the composition comprises one or more additional enzymes. For example, in some embodiments, the composition comprises one or more of a hydrolase, an esterase, a PETase, a VHIETase, and/or a cutinase. In some embodiments, the composition comprises a plurality of esterases (e.g., PETases) described herein.

### 4.9 Reaction Mixtures

In one aspect, provided herein are reaction mixtures comprising any one or more of an (a) esterase (e.g., PETase) described herein (see, e.g., §), a fusion protein described herein (see, e.g., §), a conjugate described herein (see, e.g., § Error! Reference source not found.), a polynucleotide described herein (see, e.g., § Error! Reference source not found.), a vector described herein (see, e.g., § Error! Reference source not found.), a cell (or population of cells) described herein (see, e.g., § Error! Reference source not found.)), and/or a composition described herein (see, e.g., § Error! Reference source not found.); and any one or more of (b) a terephthalic acid ester (e.g., a mono-, di-, or poly-terephthalic acid ester) (e.g., polyethylene terephthalate (PET)), a polyester (e.g., PET), and/or a polyester containing product (e.g., a plastic product comprising a polyester (e.g., PET)).

### 4.10 Reactors

In one aspect, provided herein are reactors comprising any one or more of an (a) an esterase (e.g., PETase) described herein (see, e.g., §), a fusion protein described herein (see, e.g., §), a conjugate described herein (see, e.g., § Error! Reference source not found.), a polynucleotide described herein (see, e.g., § Error! Reference source not found.), a vector described herein (see, e.g., § Error! Reference source not found.), a cell (or population of cells) described herein (see, e.g., § Error! Reference source not found.)), and/or a composition described herein (see, e.g., § Error! Reference source not found.). In some embodiments, the reactor further comprises any one or more of a terephthalic acid ester (e.g., a mono-, di-, or poly-terephthalic acid ester) (e.g., polyethylene terephthalate (PET)), a polyester (e.g., PET), and/or a polyester containing product (e.g., a plastic product comprising a polyester (e.g., PET)). In one aspect, provided herein are reactors comprising a reaction mixture described herein.

Reactors include those utilized in conventional plastic (e.g., PET) recycling technologies, such as glass or stainless-steel reactors. The volume of the reactor can be tailored to the scale needed, e.g., lab scale, pilot scale, industrial scale. In some embodiments, the reactor comprises one or more agitation or stirring devices to assist with movement and mixing of reagents with the reactor.

### 4.11 Immobilization Substrate

In some embodiments, esterase (e.g., PETase) described herein (see, e.g., §), a fusion protein described herein (see, e.g., §), a conjugate described herein (see, e.g., § Error! Reference source not found.) is immobilized on a substrate. Suitable substrates and techniques for immobilization are known to those skilled in the art. For example, the esterase (e.g., PETase) may be immobilized on a support resin by ion exchange, adsorption (e.g., hydrophobic adsorption), or covalent coupling.

In some embodiments, the substrate is a support resin. In some embodiments, the substrate is an ion exchange resin. Those skilled in the art will be familiar with the general principle of enzymatic immobilization technology and that principle can advantageously be applied in the context of immobilizing the esterase (e.g., PETase) on a substrate in accordance with the present disclosure. Suitable ion-exchange resins for immobilizing the esterase will generally comprise a polymer matrix or a polymer/ceramic hybrid matrix. An example of such a resin includes, but is not limited to, CM Ceramic HyperD® Ion Exchange Chromatography Resin. In one embodiment, the ion exchange resin is a cationic exchange resin.

For example, the esterase (e.g., a PETase) in a reaction mixture described herein, a composition described herein, or a reactor described herein may be immobilized as described above.

### 4.12 Kits

In a one aspect, provided herein are kits comprising any one or more of an esterase (e.g., PETase) described herein (see, e.g., §), a fusion protein described herein (see, e.g., §), a conjugate described herein (see, e.g., § Error! Reference source not found.), a polynucleotide described herein (see, e.g., § Error! Reference source not found.), a vector described herein (see, e.g., § Error! Reference source not found.), a cell (or population of cells) described herein (see, e.g., § Error! Reference source not found.)), and/or a composition described herein (see, e.g., § Error! Reference source not found.). In addition, the kit may comprise a technical instruction. The technical instructions of the kit may contain information about preparation and/or use of the any of the foregoing.

Any of the kits described herein may be used in any of the methods described herein (see, e.g., §).

### 4.13 Methods of Use

The disclosure provides, inter alia, various methods of utilizing any one or more of an esterase (e.g., PETase) described herein (see, e.g., §), a fusion protein described herein (see, e.g., §), a conjugate described herein (see, e.g., § Error! Reference source not found.), a polynucleotide described herein (see, e.g., § Error! Reference source not found.), a vector described herein (see, e.g., § Error! Reference source not found.), a cell (or population of cells) described herein (see, e.g., § Error! Reference source not found.)), and/or a composition described herein (see, e.g., § Error! Reference source not found.).

By way of example, the esterases (e.g., PETases) disclosed herein are suitable for a range of applications, including industrial applications, illustrative examples of which include as additives in detergents, feed compositions (including for animal feed), textile production, electronics, and biomedical applications.

### 4.13.1 Methods of Hydrolyzing a Terephthalic Acid Ester

In one aspect, provided herein are methods of hydrolyzing a terephthalic acid ester, the method comprising contacting a terephthalic acid ester with an esterase (e.g., PETase) described herein (see, e.g., §), a fusion protein described herein (see, e.g., §), a conjugate described herein (see, e.g., § Error! Reference source not found.), a polynucleotide described herein (see, e.g., § Error! Reference source not found.), a vector described herein (see, e.g., § Error! Reference source not found.), a cell (or population of cells) described herein (see, e.g., § Error! Reference source not found.)), and/or a composition described herein (see, e.g., § Error! Reference source not found.), to thereby hydrolyze the terephthalic acid ester. In some embodiments, the method is carried out under conditions sufficient to hydrolyze the terephthalic acid ester. In some embodiments, the method is carried out under conditions sufficient to convert the terephthalic acid ester to any one or more of terephthalic acid, ethylene glycol, mono-(2-hydroxyethyl) terephthalate, and/or bis-(2-hydroxyethyl) terephthalate.

In some embodiments, the terephthalic acid ester is a mono-terephthalic acid ester, a di-terephthalic acid ester, or a poly-terephthalic acid ester.

In some embodiments, the terephthalic acid ester is a polyester. In some embodiments, the polyester comprises polyethylene terephthalate (PET), polylactic acid (PLA), polytrimethylene terephthalate (PTT), polybutylene terephthalate (PBT), polyethylene isosorbide terephthalate (PEIT), polyethylene terephthalate (PET), polyhydroxyalkanoate (PHA), polybutylene succinate (PBS), polybutylene succinate adipate (PBSA), polybutylene adipate terephthalate (PBAT), polyethylene furanoate (PEF), polycapro lactone (PCL), polyethylene adipate (PEA), poly(glycolic acid) (PGA), poly(lactic-co-glycolic acid) (PLGA), or any combination of any of the foregoing. In some embodiments, the polyester comprises PET.

In some embodiments, the hydrolysis results in the conversion of the terephthalic acid ester to terephthalic acid, mono-(2-hydroxyethyl) terephthalate (MHET), bis-(2-hydroxyethyl) terephthalate (BHET), and/or and ethylene glycol (EG). In some embodiments, the hydrolysis results in the production of any one or more of terephthalic acid (TPA), mono-(2-hydroxyethyl) terephthalate (MHET), bis-(2-hydroxyethyl) terephthalate (BHET), and/or and ethylene glycol (EG). In some embodiments, the hydrolysis results in the production of mono-(2-hydroxyethyl) terephthalate (MHET). A single type of product or several different types of products may be recovered, depending on the starting substrate. In some embodiments, the method further comprises recovering, isolating, and/or purifying the MHET, BHET, TPA, and/or EG, or any combination thereof.

### 4.13.2 Methods of Hydrolyzing PET

In one aspect, provided herein are methods of hydrolyzing a polyethylene terephthalate (PET), the method comprising contacting a PET with an esterase (e.g., PETase) described herein (see, e.g., §), a fusion protein described herein (see, e.g., §), a conjugate described herein (see, e.g., § Error! Reference source not found.), a polynucleotide described herein (see, e.g., § Error! Reference source not found.), a vector described herein (see, e.g., § Error! Reference source not found.), a cell (or population of cells) described herein (see, e.g., § Error! Reference source not found.)), and/or a composition described herein (see, e.g., § Error! Reference source not found.), to thereby hydrolyze the PET. In some embodiments, the method is carried out under conditions sufficient to hydrolyze the PET. In some embodiments, the method is carried out under conditions sufficient to convert the PET to any one or more of terephthalic acid (TPA), ethylene glycol, mono-(2-hydroxyethyl) terephthalate, and/or bis-(2-hydroxyethyl) terephthalate.

In some embodiments, the PET is part of a polyester. In some embodiments, the polyester additionally comprises polylactic acid (PLA), polytrimethylene terephthalate (PTT), polybutylene terephthalate (PBT), polyethylene isosorbide terephthalate (PEIT), polyethylene terephthalate (PET), polyhydroxyalkanoate (PHA), polybutylene succinate (PBS), polybutylene succinate adipate (PBSA), polybutylene adipate terephthalate (PBAT), polyethylene furanoate (PEF), polycapro lactone (PCL), polyethylene adipate (PEA), poly(glycolic acid) (PGA), poly(lactic-co-glycolic acid) (PLGA), or any combination of any of the foregoing.

In some embodiments, the PET is part of a mixture or blend of more than one material. Additional materials include, for example, but are not limited to, metal, ceramic, glass, wood and polymers (e.g., a polyamide (e.g., nylon), polyolefin (e.g., polyethylene and polypropylene), or polyvinylchloride (PVC)). In some embodiments, the PET is selectively hydrolyzed. In some embodiments, the PET is an admixture with one or more co-material (e.g., as a melt blend or laminated structure).

In some embodiments, the hydrolysis results in the conversion of the PET to any one or more of terephthalic acid (TPA), mono-(2-hydroxyethyl) terephthalate (MHET), bis-(2-hydroxyethyl) terephthalate (BHET), and/or and ethylene glycol (EG). In some embodiments, the hydrolysis results in the production of terephthalic acid (TPA), mono-(2-hydroxyethyl) terephthalate (MHET), bis-(2-hydroxyethyl) terephthalate (BHET), and/or and ethylene glycol (EG) (or any combination thereof). In some embodiments, the hydrolysis results in the production of mono-(2-hydroxyethyl) terephthalate (MHET). A single type of product or several different types of products may be recovered, depending on the starting substrate. In some embodiments, the method further comprises recovering, isolating, and/or purifying the MHET, BHET, TPA, and/or EG, or any combination thereof.

In some embodiments, the method further comprises simultaneously or sequentially (e.g., before and/or after) contacting the PET with one or more of a mono-(2-hydroxyethyl) terephthalate hydrolase (MHETase), a bis-2-hydroxyethyl terephthalate hydrolase (BHETase), an esterase, a PETase, a carboxylesterase, and/or a cutinase.

In some embodiments, the method further comprises simultaneously or sequentially (e.g., before and/or after) contacting the PET with a mono-(2-hydroxyethyl) terephthalate hydrolase (MHETase). In some embodiments, MHET is degraded into terephthalic acid (TPA) and/or EG. In some embodiments, the method further comprises simultaneously or sequentially contacting the PET with a bis-2-hydroxyethyl terephthalate hydrolase (BHETase). In some embodiments, BHET is degraded into MHET.

### 4.13.3 Methods of Degrading a Polyester

In one aspect, provided herein are methods of degrading a polyester, the method comprising contacting a polyester with an esterase (e.g., PETase) described herein (see, e.g., §), a fusion protein described herein (see, e.g., §), a conjugate described herein (see, e.g., § Error! Reference source not found.), a polynucleotide described herein (see, e.g., § Error! Reference source not found.), a vector described herein (see, e.g., § Error! Reference source not found.), a cell (or population of cells) described herein (see, e.g., § Error! Reference source not found.)), and/or a composition described herein (see, e.g., § Error! Reference source not found.), to thereby degrade the polyester. In some embodiments, the method is carried out under conditions sufficient to degrade the polyester. In some embodiments, the method is carried out under conditions sufficient to convert the polyester to any one or more of terephthalic acid, ethylene glycol, mono-(2-hydroxyethyl) terephthalate, and/or bis-(2-hydroxyethyl) terephthalate.

In some embodiments, the polyester comprises polyethylene terephthalate (PET), polylactic acid (PLA), polytrimethylene terephthalate (PTT), polybutylene terephthalate (PBT), polyethylene isosorbide terephthalate (PEIT), polyethylene terephthalate (PET), polyhydroxyalkanoate (PHA), polybutylene succinate (PBS), polybutylene succinate adipate (PBSA), polybutylene adipate terephthalate (PBAT), polyethylene furanoate (PEF), polycapro lactone (PCL), polyethylene adipate (PEA), poly(glycolic acid) (PGA), poly(lactic-co-glycolic acid) (PLGA), or any combination of any of the foregoing. In some embodiments, the polyester comprises PET.

In some embodiments, the polyester is part of a mixture or blend of more than one material. Additional materials include, for example, but are not limited to, metal, ceramic, glass, wood and polymers (e.g., a polyamide (e.g., nylon), polyolefin (e.g., polyethylene and polypropylene), or polyvinylchloride (PVC)). In some embodiments, the polyester is selectively hydrolyzed. In some embodiments, the polyester is an admixture with one or more co-material (e.g., as a melt blend or laminated structure).

In some embodiments, the degradation results in the conversion of the polyester to any one or more of terephthalic acid (TPA), mono-(2-hydroxyethyl) terephthalate (MHET), bis-(2-hydroxyethyl) terephthalate (BHET), and/or and ethylene glycol (EG). In some embodiments, the degradation results in the production of any one or more of terephthalic acid (TPA), mono-(2-hydroxyethyl) terephthalate (MHET), bis-(2-hydroxyethyl) terephthalate (BHET), and/or and ethylene glycol (EG) (or any combination thereof). In some embodiments, the hydrolysis results in the production of mono-(2-hydroxyethyl) terephthalate (MHET). A single type of product or several different types of products may be recovered, depending on the starting substrate. In some embodiments, the method further comprises recovering, isolating, and/or purifying the MHET, BHET, TPA, and/or EG, or any combination thereof.

In some embodiments, the method further comprises simultaneously or sequentially (e.g., before and/or after) contacting the polyester with one or more of a mono-(2-hydroxyethyl) terephthalate hydrolase (MHETase), a bis-2-hydroxyethyl terephthalate hydrolase (BHETase), an esterase, a PETase, a carboxylesterase, and/or a cutinase.

In some embodiments, the method further comprises simultaneously or sequentially (e.g., before and/or after) contacting the polyester with a mono-(2-hydroxyethyl) terephthalate hydrolase (MHETase). In some embodiments, MHET is degraded into terephthalic acid (TPA) and/or EG. In some embodiments, the method further comprises simultaneously or sequentially contacting the polyester with a bis-2-hydroxyethyl terephthalate hydrolase (BHETase). In some embodiments, BHET is degraded into MHET.

4.13.4 Methods of Degrading a Plastic Product comprising a Polyester

In one aspect, provided herein are methods of degrading a plastic product comprising a polyester, the method comprising the plastic product with an esterase (e.g., PETase) described herein (see, e.g., §), a fusion protein described herein (see, e.g., §), a conjugate described herein (see, e.g., § Error! Reference source not found.), a polynucleotide described herein (see, e.g., § Error! Reference source not found.), a vector described herein (see, e.g., § Error! Reference source not found.), a cell (or population of cells) described herein (see, e.g., § Error! Reference source not found.)), and/or a composition described herein (see, e.g., § Error! Reference source not found.), to thereby degrade the plastic product. In some embodiments, the method is carried out under conditions sufficient to degrade the plastic product. In some embodiments, the method is carried out under conditions sufficient to convert the polyester in the plastic product to any one or more of terephthalic acid, ethylene glycol, mono-(2-hydroxyethyl) terephthalate, and/or bis-(2-hydroxyethyl) terephthalate.

In some embodiments, the polyester comprises polyethylene terephthalate (PET), polylactic acid (PLA), polytrimethylene terephthalate (PTT), polybutylene terephthalate (PBT), polyethylene isosorbide terephthalate (PEIT), polyethylene terephthalate (PET), polyhydroxyalkanoate (PHA), polybutylene succinate (PBS), polybutylene succinate adipate (PBSA), polybutylene adipate terephthalate (PBAT), polyethylene furanoate (PEF), polycapro lactone (PCL), polyethylene adipate (PEA), poly(glycolic acid) (PGA), poly(lactic-co-glycolic acid) (PLGA), or any combination of any of the foregoing. In some embodiments, the polyester comprises PET.

In some embodiments, the degradation results in the production of terephthalic acid (TPA), mono-(2-hydroxyethyl) terephthalate (MHET), bis-(2-hydroxyethyl) terephthalate (BHET), and/or and ethylene glycol (EG). In some embodiments, the degradation results in the production of mono-(2-hydroxyethyl) terephthalate (MHET). Monomers (e.g., any one or more of MHET, BHET, TPA, and/or EG) resulting from the depolymerization or degradation may be suitably recovered, sequentially, or continuously. In some embodiments, the hydrolysis results in the conversion of the terephthalic acid ester to terephthalic acid (TPA), mono-(2-hydroxyethyl) terephthalate (MHET), bis-(2-hydroxyethyl) terephthalate (BHET), and/or and ethylene glycol (EG).

In some embodiments, the method further comprises simultaneously or sequentially (e.g., before and/or after) contacting the plastic product with one or more of a mono-(2-hydroxyethyl) terephthalate hydrolase (MHETase), a bis-2-hydroxyethyl terephthalate hydrolase (BHETase), an esterase, a PETase, a carboxylesterase, and/or a cutinase.

In some embodiments, the method further comprises simultaneously or sequentially (e.g., before and/or after) contacting the plastic product with a mono-(2-hydroxyethyl) terephthalate hydrolase (MHETase). In some embodiments, MHET is degraded into terephthalic acid (TPA) and/or EG. In some embodiments, the method further comprises simultaneously or sequentially contacting the plastic product with a bis-2-hydroxyethyl terephthalate hydrolase (BHETase). In some embodiments, BHET is degraded into MHET.

In some embodiments, the plastic product is subject to thermal processing, washing and/or mechanical grinding prior to the contacting with the esterase (e.g., PETase), fusion protein, conjugate, polynucleotide, vector, cell (or population of cells), composition. In some embodiments, the thermal processing renders a crystalline or semi-crystalline structure of the polyester (e.g., PET) amorphous. In some embodiments, plastic product is pre-treated prior to be contacted with the esterase (e.g., PETase), fusion protein, conjugate, polynucleotide, vector, cell (or population of cells), or composition in order to physically change its structure, so as to increase the surface of contact between the polyester and the esterase (e.g., PETase), fusion protein, conjugate, polynucleotide, vector, cell (or population of cells), or composition.

In some embodiments, the plastic product comprises a textile, container (e.g., bag, bottle), film, or powder.

4.13.5 Methods of Recycling a Plastic Product comprising a Polyester

In one aspect, provided herein are methods of recycling a plastic product comprising a polyester, the method comprising the plastic product with an esterase (e.g., PETase) described herein (see, e.g., §), a fusion protein described herein (see, e.g., §), a conjugate described herein (see, e.g., § Error! Reference source not found.), a polynucleotide described herein (see, e.g., § Error! Reference source not found.), a vector described herein (see, e.g., § Error! Reference source not found.), a cell (or population of cells) described herein (see, e.g., § Error! Reference source not found.)), and/or a composition described herein (see, e.g., § Error! Reference source not found.), to thereby recycle the plastic product. In some embodiments, the method is carried out under conditions sufficient to recycle the plastic product. In some embodiments, the method is carried out under conditions sufficient to convert the polyester in the plastic product to any one or more of terephthalic acid, ethylene glycol, mono-(2-hydroxyethyl) terephthalate, and/or bis-(2-hydroxyethyl) terephthalate.

In some embodiments, the polyester comprises polyethylene terephthalate (PET), polylactic acid (PLA), polytrimethylene terephthalate (PTT), polybutylene terephthalate (PBT), polyethylene isosorbide terephthalate (PEIT), polyethylene terephthalate (PET), polyhydroxyalkanoate (PHA), polybutylene succinate (PBS), polybutylene succinate adipate (PBSA), polybutylene adipate terephthalate (PBAT), polyethylene furanoate (PEF), polycapro lactone (PCL), polyethylene adipate (PEA), poly(glycolic acid) (PGA), poly(lactic-co-glycolic acid) (PLGA), or any combination of any of the foregoing. In some embodiments, the polyester comprises PET.

In some embodiments, the degradation results in the production of any one or more of terephthalic acid (TPA), mono-(2-hydroxyethyl) terephthalate (MHET), bis-(2-hydroxyethyl) terephthalate (BHET), and/or and ethylene glycol (EG) (or any combination thereof). In some embodiments, the degradation results in the production of mono-(2-hydroxyethyl) terephthalate (MHET). Any of the products (e.g., any one or more of MHET, BHET, TPA, and/or EG) resulting from the depolymerization or degradation may be suitably recovered, sequentially, or continuously. A single type of product or several different types of products may be recovered, depending on the starting substrate. In some embodiments, the method further comprises recovering, isolating, and/or purifying the MHET, BHET, TPA, and/or EG, or any combination thereof.

In some embodiments, the method further comprises simultaneously or sequentially (e.g., before and/or after) contacting the plastic product with one or more of a mono-(2-hydroxyethyl) terephthalate hydrolase (MHETase), a bis-2-hydroxyethyl terephthalate hydrolase (BHETase), an esterase, a PETase, a carboxylesterase, and/or a cutinase.

In some embodiments, the method further comprises simultaneously or sequentially (e.g., before and/or after) contacting the plastic product with a mono-(2-hydroxyethyl) terephthalate hydrolase (MHETase). In some embodiments, MHET is degraded into terephthalic acid (TPA) and/or EG. In some embodiments, the method further comprises simultaneously or sequentially contacting the plastic product with a bis-2-hydroxyethyl terephthalate hydrolase (BHETase). In some embodiments, BHET is degraded into MHET.

In some embodiments, the plastic product is subject to thermal processing, washing and/or mechanical grinding prior to the contacting with the esterase (e.g., PETase), fusion protein, conjugate, polynucleotide, vector, cell (or population of cells), composition. In some embodiments, the thermal processing renders a crystalline or semi-crystalline structure of the polyester (e.g., PET) amorphous. In some embodiments, plastic product is pre-treated prior to be contacted with the esterase (e.g., PETase), fusion protein, conjugate, polynucleotide, vector, cell (or population of cells), or composition in order to physically change its structure, so as to increase the surface of contact between the polyester and the esterase (e.g., PETase), fusion protein, conjugate, polynucleotide, vector, cell (or population of cells), or composition.

In some embodiments, the plastic product comprises a textile, container (e.g., bag, bottle), film, or powder.

4.13.6 Methods of Recovering, Isolating, and/or Purifying Cleavage Products

In one aspect, provided herein are method of recovering, isolating, and/or purifying any one or more of the IHET, BHET, TPA, and/or EG, or any combination thereof, produced by any of the foregoing methods, e.g., any one of the methods described in §§, or Error! Reference source not found. In some embodiments, the hydrolysis results in the conversion of the terephthalic acid ester to terephthalic acid (TPA), mono-(2-hydroxyethyl) terephthalate (MHET), bis-(2-hydroxyethyl) terephthalate (BHET), and/or and ethylene glycol (EG).

The recovered products may be further purified, using any suitable purifying method and conditioned in a repolymerizable form. Illustrative examples of suitable purifying methods include stripping process, separation by aqueous solution, steam selective condensation, filtration and concentration of the medium after the bioprocess, separation, distillation, vacuum evaporation, extraction, electrodialysis, adsorption, ion exchange, precipitation, crystallization, concentration and acid addition dehydration and precipitation, nanofiltration, acid catalyst treatment, semi continuous mode distillation or continuous mode distillation, solvent extraction, evaporative concentration, evaporative crystallization, liquid/liquid extraction, hydrogenation, azeotropic distillation process, adsorption, column chromatography, simple vacuum distillation and microfiltration, combined or not.

The repolymerizable products may be used to synthesize new polyesters. In some embodiments, polyesters of same nature are repolymerized. It is further possible to mix the recovered products with other products, for example, in order to synthesize new copolymers. Alternatively, the recovered products may be used as chemical intermediates in order to produce new chemical compounds of interest.

### 4.13.7 Composition Comprising the Recovered Cleavage Products

In one aspect, provided herein are compositions comprising the recovered MHET, BHET, TPA, and/or EG, or any combination thereof described in §. In some embodiments, the composition comprises PET. In some embodiments, the composition is a plastic product. In some embodiments, the plastic product comprises PET. In some embodiments the plastic product comprises a textile, container (e.g., bag, bottle), film, or powder.

In one aspect, further provided herein are masterbatch compositions comprising esterase (e.g., PETase), composition, or cell expressing the esterase (e.g., PETase) or an extract thereof containing the esterase (e.g., PETase). Such plastic compound or masterbatch composition described herein can be used for the production of a polyester containing material. In some embodiments, the resulting plastic compound or masterbatch composition is a biodegradable plastic compound or masterbatch composition complying with at least one of the relevant standards and/or labels known by the person skilled in the art, such as standard EN 13432, standard ASTM D6400, OK Biodegradation Soil (Label Vincotte), OK Biodegradation Water (Label Vincotte), OK Compost (Label Vincotte), OK Home Compost (Label Vincotte).

### 4.13.8 Methods of Producing PET & Plastic Products

In one aspect, provided herein are methods of utilizing the MHET, BHET, TPA, and/or EG, or any combination thereof, produced by any one of the methods described herein (e.g., any one of the methods described in §§, or Error! Reference source not found.) and/or recovered by a method described herein (as described in § Error! Reference source not found.), to produce a PET. In some embodiments, the method comprises a polycondensation process.

In one aspect, provided herein are methods of utilizing the MHET, BHET, TPA, and/or EG, or any combination thereof, produced by any one of the methods described herein (e.g., any one of the methods described in §§, or Error! Reference source not found.) and/or recovered by a method described herein (as described in § Error! Reference source not found.), to produce a plastic product. In some embodiments, the method comprises a polycondensation process.

In some embodiments, the plastic product comprises a polyester. In some embodiments, the polyester comprises polyethylene terephthalate (PET), polylactic acid (PLA), polytrimethylene terephthalate (PTT), polybutylene terephthalate (PBT), polyethylene isosorbide terephthalate (PEIT), polyethylene terephthalate (PET), polyhydroxyalkanoate (PHA), polybutylene succinate (PBS), polybutylene succinate adipate (PBSA), polybutylene adipate terephthalate (PBAT), polyethylene furanoate (PEF), polycapro lactone (PCL), polyethylene adipate (PEA), poly(glycolic acid) (PGA), poly(lactic-co-glycolic acid) (PLGA), or any combination of any of the foregoing. In some embodiments, the polyester comprises PET. In some embodiments, the plastic product comprises PET.

In some embodiments the plastic product comprises a textile, container (e.g., bag, bottle), film, or powder.

### 5. EXAMPLES

### 5.1 Example 1. Esterase Expression and Purification

HIS tagged versions of each of esterases (e.g., PETases) 1-11 (SEQ ID NOS: 2-12) and reference esterase 1 (SEQ ID NO: 1) were expressed and purified according to standard methods known in the art. The HIS tagged version comprised a MGSSH IH (SEQ ID NO: 71) peptide or a MGSSHHHH IHHH (SEQ ID NO: 72) peptide fused to the terminus of the esterase. Briefly, plasmids were designed comprising a codon optimized coding sequence for each of the esterases (e.g., PETases), a T7 promoter to drivee protein expression in bacteria, a transcription termination signal, and a carbenicillin-resistance gene. Each of the generated plasmids was transformed into a BL21(DE3) competent variant of E. coli (C2527H, New England Biolabs) using a standard transformation protocol. Following transformation, the E. coli cells were spread on carbenicillin plates and maintained at 37 °C overnight, selectively facilitating the growth of transformed colonies. A single colony was then selected from the plate and inoculated into a culture tube containing 5 mL of LB medium supplemented with carbenicillin antibiotic. The culture tube, was subsequently placed in a shaking incubator set at 800 rpm and held at a consistent 37 °C for 3-4 hours. Upon achieving an optical density between 0.4 and 0.8, the samples were incubated at 20 °C with shaking (constant rpm) for 20 hours. Following incubation, the culture tubes were centrifuged for 20 minutes at 4000×g to pellet bacterial cells. The separated supernatant was discarded, and the cell pellet resuspended in sonication buffer. The cells were lysed with sonication using a Branson Sonifier. The proteins were purified using standard His tag purification as detailed by Spriestersbach A, Kubicek J, Schafer F, Block H, Maertens B. Purification of His-Tagged Proteins. Methods Enzymol. 2015; 559:1-15. doi: 10.1016/bs.mie.2014.11.003. Epub 2015 May 4. PMID: 26096499 (the entire contents of which are incorporated herein by reference for all purposes).

### 5.2 Example 2. Esterase (e.g., PETase) Activity

The esterase (e.g., PETase) activity of each of the HIS tagged esterases (e.g., PETases) 1-11 (SEQ ID NOS: 2-12) generated in Example 1 was assessed using standard methods known in the art. Briefly, coupons (approx. 8 mg) of amorphous PET film (Goodfellow, ES30-FM-000145) was transferred to specific wells of a 96 well polystyrene plate. Typical reaction mixtures were assembled by combining the following components in the following order: MilliQ water (140 μl), and 460 mM HEPES buffer pH 8 with 2% (w/v) NaCl (60 μl), and 0.5 μM purified enzyme stock solution (50 μl). Purified enzyme (esterase (e.g., PETase)) stocks were prepared in 5 mM HEPES buffer pH 8 with 0.5% NaCl (referred to as Enzyme Diluent). In the no-enzyme negative controls, Enzyme Diluent was added in place of the enzyme stock. In the no-substrate controls, the reaction mixture was added to a well lacking the PET substrate coupon (the amorphous PET film). In some experiments the final enzyme concentration was increased 20-fold by reducing the MilliQ water volume (5 μl) and replacing the 0.5 μM enzyme stock with 2.7 μM enzyme stock (185 μl) in Enzyme Diluent.

The plate containing the combined reactions was sealed with a foil film to prevent evaporation and incubated at 50 °C for 24 hours. Following this incubation, aliquots (90 μl) of each reaction mixture were transferred to a UV-transparent 96 well plate. The following components were added to each well containing sample aliquots: MilliQ water (22.5 μl) and dimethyl sulfoxide (DMSO, 12.5 μl). DMSO was added to match the composition of standards containing known amounts of bis-2-hydroxyethyl terephthalate (BHET). A Clariostar BMG plate reader was used to measure absorbance at 290 nm for samples and standards. Standards were used to generate calibration curves and quantify the aromatic compounds solubilized following incubation with PETase enzymes. The quantification data is set forth below in Table 4 for each of the tested HIS tagged esterases (e.g., PETases) (Esterase1-11 (SEQ ID NOS: 2-12) and Reference Esterase 1 (SEQ ID NO: 1)).

TABLE 4
Absorbance (290 nm) of Generated Soluble Aromatic Compounds.

Esterase (e.g., PETase) Concentration

| Description | 0.1 uM | 0.2 uM | 2.0 uM |
|---|---|---|---|
| Esterase-1 | >1 ≤ 10 | >10 ≤ 20 | ND |
| Esterase-2 | ND | >1 ≤ 10 | ND |
| Esterase-3 | ≤1 | >1 ≤ 10 | ND |
| Esterase-4 | ≤1 | ND | ND |
| Esterase-5 | ND | >50 ≤ 60 | ND |
| Esterase-6 | ≤1 | >20 ≤ 30 | >20 ≤ 30 |
| Esterase-7 | ND | >30 ≤ 40 | ND |
| Esterase-8 | ND | >1 ≤ 10 | >20 ≤ 30 |
| Esterase-9 | ND | >30 ≤ 40 | >80 ≤ 90 |
| Esterase-10 | ND | >1 ≤ 10 | ND |
| Esterase-11 | ND | >30 ≤ 40 | >10 ≤ 20 |
| Esterase | >1 ≤ 10 | ND | ND |
| Reference 1 | | | |

*ND = Not Determined

As shown in Table 4, each of the tested esterases (e.g., PETases) exhibited esterase (e.g., PETase) activity demonstrated by the presence of soluble aromatic compounds (e.g., MHET) liberated by the breakdown of polyethylene terephthalate (PET) fragments.

1. A recombinant esterase that comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any esterase set forth in any one of SEQ ID NOS: 2-12.

2.-3. (canceled)

4. A recombinant esterase that comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, identical to the amino acid sequence set forth in SEQ ID NO: 1 and comprises one or more amino acid variation relative to the amino acid sequence set forth in SEQ ID NO: 1.

5. The recombinant esterase of claim 4, comprising an amino acid variation at one or more of amino acid positions S11, T14, A18, A21, T22, A23, F41, K43, D47, S48, V50, D77, M91, M93, E96, S107, E111, A118, P126, K130, 1157, N158, A172, A180, L183, S186, A194, A206, R203, S207, Y216, M235, F247, S249, S252, and/or G262 relative to the amino acid sequence set forth in SEQ ID NO: 1.

6.-12. (canceled)

13. A fusion protein comprising the esterase of claim 1 operably connected to a heterologous protein.

14. (canceled)

15. A conjugate comprising the esterase of claim 1 operably connected to a heterologous moiety.

16. A nucleic acid molecule encoding the esterase of claim 1.

17. (canceled)

18. A vector comprising the nucleic acid molecule of claim 16.

19. (canceled)

20. A cell comprising the esterase of claim 1 or a nucleic acid molecule encoding the same.

21. A cell genetically modified to express the esterase of claim 1.

22.-25. (canceled)

26. A composition comprising the esterase of claim 1.

27.-28. (canceled)

29. A reaction mixture comprising
(a) the esterase of claim 1; and
(b) a terephthalic acid ester, a polyester or a plastic product comprising a polyester.

30. A reactor vessel comprising the esterase of claim 1.

31. A kit comprising the esterase of claim 1; and optionally instructions for using any one or more of the foregoing.

32. A method of producing the esterase of claim 1, the method comprising:
a) introducing a nucleic acid molecule encoding the esterase of claim 1 into a host cell;
b) culturing the host cell under conditions and for an amount of time suitable to allow expression of the esterase; and optionally
c) isolating and/or purifying the esterase.

33. A method of hydrolyzing a terephthalic acid ester, the method comprising contacting a terephthalic acid ester with the esterase of claim 1, to thereby hydrolyze the terephthalic acid ester.

34.-39. (canceled)

40. A method of hydrolyzing a polyethylene terephthalate (PET), the method comprising contacting a PET with the esterase of claim 1, to thereby hydrolyze the PET.

41.-43. (canceled)

44. A method of hydrolyzing a polyester, the method comprising contacting a polyester with the esterase of claim 1, to thereby hydrolyze the polyester.

45.-47. (canceled)

48. A method of degrading a plastic product comprising a polyester, the method comprising contacting the plastic product with the esterase of claim 1, to thereby degrade the plastic product.

49.-52. (canceled)

53. A method of recycling a plastic product comprising a polyester, the method comprising contacting the plastic product with the esterase of claim 1, to thereby recycle the plastic product.

54.-59. (canceled)

60. A method of producing PET or a plastic product, the method comprising utilizing the MHET, BHET, TPA, and/or EG, or any combination thereof, produced by the method of claim 33.

61.-70. (canceled)


SEQUENCE LISTING (72 sequences)

SEQ ID NO: 1 - protein, 275 aa, synthetic construct
FSAPGPSAPCSNCTRGPAPTATALKASTGPFSTAKFSVSGFLKGFGDSTVYYPTNTTGKMGAIAVIPGYLSYESSIDWWGPRLASHGFVVMTMNTETIYDQPDSRASQLSEALDYLIAQSGSSSSPLYNKIDSTRLGAIGWSMGGGGSLKLSTQRSINAIIPQAPWYSGSNAFNTIKTPALILACSADTVAPVAQHASPFYNRIPASTPKAFLEIYGGSHFCANSGYPNEDLLGMYGIAWMKRFIDFDSRYSQFLCGPNHEGDLAISEYRQNCPY

SEQ ID NO: 2 - protein, 275 aa, synthetic construct
FSAPGPSAPCSNCTRGPAPTATALKASTGPFSTAKFSVSGFLKGFGDSTVYYPTNTTGKMGAIAVIPGYLSYESSIDWWGPRLASHGFVVMTMNTETIYDQPDSRASQLSEALDYLIAQSGSSSSPLYNKIDSTRLGAIGWSMGGGGSLKLSTQRSINAIIPQAPWYSGSNAFNTIKTPALILACSADTVAPVAQHASPFYNRIPASTPKAFLEINGGSHFCANSGYPNEDLLGMYGIAWMKRFIDFDSRYSQFLCGPNHEGDLAISEYRQNCPY

SEQ ID NO: 3 - protein, 275 aa, synthetic construct
FSAPGPSAPCSNCTRGPAPTATALKASTGPFSTAKFSVSGFLKGFGDSTVYYPTNTTGKMGAIAVIPGYLSYESSIDWWGPRLASHGFVVMTMNTETIYDQPDSRASQLSEALDYLIAQSGSSSSPLYNKIDSTRLGAIGWSMGGGGSLKLSTQRSINAIIPQAPWYSGSNSFNTIKTPALILACSADTVAPVAQHASPFYNRIPASTPKAFLEIYGGSHFCANSGYPNEDLLGMYGIAWMKRFIDFDSRYSQFLCGPNHEGDLAISEYRQNCPY

SEQ ID NO: 4 - protein, 275 aa, synthetic construct
FSAPGPSAPCSNCTRGPAPTATALKASTGPFSTAKFSVSGFLKGFGDSTVYYPTNTTGKMGAIAVIPGYLSYESSIDWWGPRLASHGFVVMTMNTETIYDQPDSRASQLSEALDYLIAQSGSSSSPLYNKIDSTRLGAIGWSMGGGGSLKLSTQRSINAIIPQAPWYSGSNAFNTIKTPALILACSADTVAPVAQHASPFYNRIPASTPKAFLEIYGGSHFCANSGYPNEDLLGMYGIAWMKRFIDNDSRYSQFLCGPNHEGDLAISEYRQNCPY

SEQ ID NO: 5 - protein, 275 aa, synthetic construct
FSAPGPSAPCSNCTRGPAPTATALKASTGPFSTAKFSVSGFLKGFGDSTVYYPTNTTGKMGAIAVIPGYLSYESSIDWWGPRLASHGFVVMTMNTETIYDQPDSRASQLSEALDYLIAQSGSSSSPLYNKIDSTRLGAIGWSMGGGGSLKLSTQRSIKAIIPQAPWYSGSNAFNTIKTPALILACSADTVAPVAQHASPFYNRIPASTPKAFLEIYGGSHFCANSGYPNEDLLGMYGIAWMKRFIDFDSRYSQFLCGPNHEGDLAISEYRQNCPY

SEQ ID NO: 6 - protein, 275 aa, synthetic construct
FSAPGPSAPCSNCTRGPAPTATALKASTGPFSTAKFSVSGFLKGFGDSTVYYPTNTTGKMGAIAVIPGYLSYESSIDWWGPRLASHGFVVMTMNTETIYDQPDSRASQLSEALDYLIAQSGSSSSPLYNKIDSTRLGAIGWSMGGGGSLKLSTQRSINAIIPQAPWYSGSNAFNTIKTPALILACSADTVAPVAQHASPFYNNIPASTPKAFLEIYGGSHFCANSGYPNEDLLGMYGIAWMKRFIDFDSRYSQFLCGPNHEGDLAISEYRQNCPY

SEQ ID NO: 7 - protein, 275 aa, synthetic construct
FSAPGPSAPCSNCTRGPAPTATALKASTGPFSTAKFSVSGFLKGFGDGTVYYPTNTTGKMGAIAVIPGYLSYESSIDWWGPRLASHGFVVMTMNTETIYDQPDSRASQLSEALDYLIAQSGSSSSPLYNKIDSTRLGAIGWSMGGGGSLKLSTQRSINAIIPQAPWYSGSNAFNTIKTPALILACSADTVAPVAQHASPFYNRIPASTPKAFLEIYGGSHFCANSGYPNEDLLGMYGIAWMKRFIDFDSRYSQFLCGPNHEGDLAISEYRQNCPY

SEQ ID NO: 8 - protein, 275 aa, synthetic construct
FSAPGPSAPCSNCTRGPNPTVTALKASTGPFSTAKFSVSGFLKGFGDSTVYYPTNTTGKMGAIAVIPGYLSYESSIDWWGPRLASHGFVVITINTNTIYDQPDSRASQLSEALDYLIAQSGSSSSPLYNMIDSTRLGAIGWSMGGGGSLKLSTQRSLNAIIPQAPWYSGSNAFNTIKTPTLILACSADTVAPVSQHASPFYNRIPGTTPKAFLEIYGGSHFCANSGYPNEDLLGMYGIAWMKRFIDFDTRYSQFLCGPNHEGDLAISEYRQNCPY

SEQ ID NO: 9 - protein, 275 aa, synthetic construct
FSAPGPSAPCTNCTRGPAPTATALKASTGPFSTAKFSVSGFLKGFGDSTIYYPTNTTGKMGAIAVIPGYLSYESSIEWWGPRLASHGFVVMTMNTETIYDQPDSRASQLSEALDYLITQSGSSSSPLYNKIDSTRLGAIGWSMGGGGSLKLSTQRSINAIIPQAPWYSGSNAFNTIKTPTLILACQADTVAPVAQHASPFYNRIPASTPKAFLEIYGGSHFCANSGYPNEDLLGMYGIAWMKRFIDFDTRYSQFLCGPNHEGDLAISEYRQNCPY

SEQ ID NO: 10 - protein, 275 aa, synthetic construct
FSAPGPSAPCSNCERGPNPTEASLKASTGPFSTAKFSVSGLLSGFGGSTVYYPTNTTGKMGAIAVIPGYLSYESSIEWWGPRLASHGFVVMTINTNTIYDQPDSRANQLSAALDYLIAQSGSSSSALYNKIDSTRLGAIGWSMGGGGSLKLSTQRSINAIIPQAPWYSGSNSFNTIKTPTLIIACSADTVAPVAQHASPFYNRIPDSTPKAFLEINGGSHFCANSGYPNEDLLGKYGIAWMKRFIDFDTRYDQFLCGPNHESDLAISEYRQNCPY

SEQ ID NO: 11 - protein, 275 aa, synthetic construct
FSAPGPSAPCSNCTRGPAPTATALKASTGPFSTAKFSVSGFLKGFGDSTVYYPTNTTGKMGAIAVIPGYLSYESSIDWWGPRLASHGFVVMTMNTETIYDQPDSRASQLSEALDYLIAQSGSSSSPLYNKIDSTRLGAIGWSMGGGGSLKLSTQRSINAIIPQAPWYSGSNAFNTIKTPALILACSADTVAPVAQHASPFYNRIPASTPKAFLEIYGGSHFCANSGYPNEDLLGMYGIAWMKRFIDFDIRYSQFLCGPNHEGDLAISEYRQNCPY

SEQ ID NO: 12 - protein, 275 aa, synthetic construct
FSAPGPSAPCSNCTRGPAPTATALKASTGPFSTAKFSVSGFLKGFGDSTVYYPTNTTGKMGAIAVIPGYLSYESSIDWWGPRLASHGFVVMTMNTETIYDQPDSRASQLSEALDYLIAQSGSSSSPLYNKIDSTRLGAIGWSMGGGGSLKLSTQRSINAIIPQAPWYSGSNAFNTIKTPALILACSADTVAPVAQHASPFYNRIPASTPKAFLEIYGGSHFCANSGYPNEDLLGKYGIAWMKRFIDFDSRYSQFLCGPNHEGDLAISEYRQNCPY

SEQ ID NO: 13 - skipped

SEQ ID NO: 14 - protein, 4 aa, synthetic construct
GGGG

SEQ ID NO: 15 - protein, 5 aa, synthetic construct
GGGGG

SEQ ID NO: 16 - protein, 6 aa, synthetic construct
GGGGGG

SEQ ID NO: 17 - protein, 7 aa, synthetic construct
GGGGGGG

SEQ ID NO: 18 - protein, 8 aa, synthetic construct
GGGGGGGG

SEQ ID NO: 19 - skipped

SEQ ID NO: 20 - protein, 6 aa, synthetic construct
GSSGSS

SEQ ID NO: 21 - protein, 9 aa, synthetic construct
GSSGSSGSS

SEQ ID NO: 22 - protein, 12 aa, synthetic construct
GSSGSSGSSGSS

SEQ ID NO: 23 - protein, 15 aa, synthetic construct
GSSGSSGSSGSSGSS

SEQ ID NO: 24 - protein, 18 aa, synthetic construct
GSSGSSGSSGSSGSSGSS

SEQ ID NO: 25 - skipped

SEQ ID NO: 26 - protein, 6 aa, synthetic construct
GGSGGS

SEQ ID NO: 27 - protein, 9 aa, synthetic construct
GGSGGSGGS

SEQ ID NO: 28 - protein, 12 aa, synthetic construct
GGSGGSGGSGGS

SEQ ID NO: 29 - protein, 15 aa, synthetic construct
GGSGGSGGSGGSGGS

SEQ ID NO: 30 - protein, 18 aa, synthetic construct
GGSGGSGGSGGSGGSGGS

SEQ ID NO: 31 - protein, 5 aa, synthetic construct
GGGGS

SEQ ID NO: 32 - protein, 10 aa, synthetic construct
GGGGSGGGGS

SEQ ID NO: 33 - protein, 15 aa, synthetic construct
GGGGSGGGGSGGGGS

SEQ ID NO: 34 - protein, 25 aa, synthetic construct
GGGGSGGGGSGGGGSGGGGSGGGGS

SEQ ID NO: 35 - protein, 30 aa, synthetic construct
GGGGSGGGGSGGGGSGGGGSGGGGSGGGGS

SEQ ID NO: 36 - protein, 6 aa, synthetic construct
GGSGGG

SEQ ID NO: 37 - protein, 6 aa, synthetic construct
GGGGGS

SEQ ID NO: 38 - protein, 6 aa, synthetic construct
GGSGSS

SEQ ID NO: 39 - protein, 6 aa, synthetic construct
GSSGGS

SEQ ID NO: 40 - skipped

SEQ ID NO: 41 - protein, 6 aa, synthetic construct
GSSGSS

SEQ ID NO: 42 - protein, 9 aa, synthetic construct
GSSGSSGSS

SEQ ID NO: 43 - protein, 12 aa, synthetic construct
GSSGSSGSSGSS

SEQ ID NO: 44 - protein, 15 aa, synthetic construct
GSSGSSGSSGSSGSS

SEQ ID NO: 45 - protein, 18 aa, synthetic construct
GSSGSSGSSGSSGSSGSS

SEQ ID NO: 46 - protein, 6 aa, synthetic construct
GGGGSS

SEQ ID NO: 47 - protein, 9 aa, synthetic construct
GGSGGGGSS

SEQ ID NO: 48 - protein, 9 aa, synthetic construct
GGSGSSGGG

SEQ ID NO: 49 - protein, 9 aa, synthetic construct
GGGGGSGSS

SEQ ID NO: 50 - protein, 9 aa, synthetic construct
GGGGSSGGS

SEQ ID NO: 51 - protein, 9 aa, synthetic construct
GSSGGSGGG

SEQ ID NO: 52 - protein, 9 aa, synthetic construct
GSSGGGGGS

SEQ ID NO: 53 - protein, 5 aa, synthetic construct
EAAAK

SEQ ID NO: 54 - protein, 10 aa, synthetic construct
EAAAKEAAAK

SEQ ID NO: 55 - protein, 15 aa, synthetic construct
EAAAKEAAAKEAAAK

SEQ ID NO: 56 - protein, 20 aa, synthetic construct
EAAAKEAAAKEAAAKEAAAK

SEQ ID NO: 57 - protein, 25 aa, synthetic construct
EAAAKEAAAKEAAAKEAAAKEAAAK

SEQ ID NO: 58 - protein, 30 aa, synthetic construct
EAAAKEAAAKEAAAKEAAAKEAAAKEAAAK

SEQ ID NO: 59 - protein, 11 aa, synthetic construct
GGSGGGEAAAK

SEQ ID NO: 60 - protein, 11 aa, synthetic construct
GGSEAAAKGGG

SEQ ID NO: 61 - protein, 11 aa, synthetic construct
GGGGGSEAAAK

SEQ ID NO: 62 - protein, 11 aa, synthetic construct
GGGEAAAKGGS

SEQ ID NO: 63 - protein, 11 aa, synthetic construct
EAAAKGGSGGG

SEQ ID NO: 64 - protein, 11 aa, synthetic construct
EAAAKGGGGGS

SEQ ID NO: 65 - skipped

SEQ ID NO: 66 - protein, 5 aa, synthetic construct
PAPAP

SEQ ID NO: 67 - protein, 7 aa, synthetic construct
PAPAPAP

SEQ ID NO: 68 - protein, 9 aa, synthetic construct
PAPAPAPAP

SEQ ID NO: 69 - protein, 11 aa, synthetic construct
PAPAPAPAPAP

SEQ ID NO: 70 - protein, 13 aa, synthetic construct
PAPAPAPAPAPAP

SEQ ID NO: 71 - protein, 10 aa, synthetic construct
MGSSHHHHHH

SEQ ID NO: 72 - protein, 12 aa, synthetic construct
MGSSHHHHHHHH